(19) United States
(12) Patent Application Publication
(10) Pub. No.: US 2025/0215464 A1
(43) Pub. Date: Jul. 3, 2025

(54) ENGINEERED ENZYME FOR PREPARING A HYDROXYLATED INDANONE INTERMEDIATE USEFUL IN THE SYNTHESIS OF BELZUTIFAN

(71) Applicant: Merck Sharp & Dohme LLC, Rahway, NJ (US)

(72) Inventors: Wai Ling Cheung-Lee, Westfield, NJ (US); Daniel A. DiRocco, Califon, NJ (US); Agnieszka Gil, Branchburg, NJ (US); Kaori Hiraga, North Bergen, NJ (US); Jungchul Kim, Basking Ridge, NJ (US); Joshua Kolev, Califon, NJ (US); Birgit Kosjek, Westfield, NJ (US); Kevin M. Maloney, Metuchen, NJ (US); Nastaran Salehi Marzijarani, Mahwah, NJ (US); John McIntosh, Brookline, MA (US); Jonathan P. McMullen, Scotch Plains, NJ (US); Jeffrey C. Moore, Westfield, NJ (US); Grant S. Murphy, Brookline, MA (US); Weilan Pan, Downington, PA (US); Juan E. Velasquez Velez, Jersey City, NJ (US); Deeptak Verma, Feasterville-Trevose, PA (US); Matthew S. Winston, Maplewood, NJ (US); Li Xiao, Cranbury, NJ (US); Victoria Zhang, Bardonia, NY (US)

(73) Assignee: Merck Sharp & Dohme LLC, Rahway, NJ (US)

(21) Appl. No.: 18/880,951

(22) PCT Filed: Jul. 5, 2023
(86) PCT No.: PCT/US2023/026887

§ 371 Date: Jan. 3, 2025

Related U.S. Application Data
(60) Provisional application No. 63/359,337, filed on Jul. 8, 2022.

(51) Int. Cl. C12P 11/00; C12N 1/20; C12N 9/02; C12N 15/70; C12R 1/19

(52) U.S. Cl. CPC C12P 11/00; C12N 1/20; C12N 9/0071; C12N 15/70; C12R 2001/19; C12Y 114/11

(57) ABSTRACT

The present disclosure provides enzymes derived from the fungi Fusarium oxysporum c8D (“FoPip4H enzymes”) having improved properties as compared to a naturally occurring wild-type enzyme including the capability of hydroxylating certain substituted indanones to provide optically pure 3-hydroxyindanones. Also provided are polynucleotides encoding the FoPip4H enzymes, host cells capable of expressing the FoPip4H enzymes, and processes for using the FoPip4H enzymes to synthesize (R)-4-fluoro-3-hydroxy-7-(methylsulfonyl)-2, 3-dihydro-1H-inden-1-one, a useful intermediate in the synthesis of belzutifan.

### FIELD OF THE INVENTION

The present invention relates provides hydroxylating enzymes derived from the fungi Fusarium oxysporum c8D, useful in biocatalytic and synthetic processes involving oxidation of an indanone to provide a chiral alcohol. Such enzymes are particularly useful in providing an optically pure 3-hydroxyindanone which is a useful intermediate in the preparation of belzutifan.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Oct. 31, 2022, is named 25554-WO-PCT_SL.XML and is 24,536 bytes in size.

### BACKGROUND OF THE INVENTION

Enzymes are polypeptides that serve to accelerate the chemical reactions of living cells (often by several orders of magnitude). Without enzymes, most biochemical reactions would be too slow to even carry out life processes. Enzymes display great specificity and are not permanently modified by their participation in reactions. Since they are not changed during the reactions, enzymes are particularly cost effective when used as catalysts for a desired chemical transformation.

M. Hibi et al. discloses a protein derived from the fungi Fusarium oxysporum c8D that is capable of hydroxylating pipecolic acid. Appl Environ Microbiol. 2016 Apr. 1; 82 (7): 2070-2077. The reference discloses that this enzyme is part of the Fe (II)/a-ketoglutarate-dependent dioxygenase superfamily. It further discloses that 4-hydroxy amino acids such as (4S)-hydroxy-L and D-pipecolic acids can be efficiently produced at high optical purities.

Recently, 3-[(1S,2S,3R)-2,3-difluoro-1-hydroxy-7-methylsulfonyl-indan-4-yl]oxy-5-fluoro-benzonitrile (hereinafter, belzutifan), a novel HIF-2α inhibitor, received U.S. Food and Drug Administration approval for the treatment of adult patients with von Hippel-Lindau (VHL) disease who require therapy for associated renal cell carcinoma (RCC), central nervous system (CNS) hemangioblastomas, or pancreatic neuroendocrine tumors (pNET), not requiring immediate surgery.

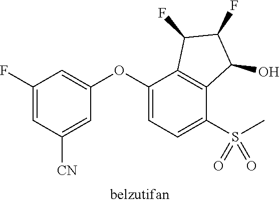 [SMILES: CS(=O)(=O)c1ccc(Oc2cc(F)cc(C#N)c2)c2c1[C@H](O)[C@H](F)[C@@H]2F]

In a recent clinical study, 49% of patients with RCC associated with VHL disease who received belzutifan had a confirmed objective response; most patients had a reduction in renal tumor size. In addition, 30% of patients with CNS hemangioblastomas had a response after treatment with belzutifan and 91% of patients with pancreatic neuroendocrine tumors responded after treatment. Belzutifan could serve as an alternative therapy or complement surgical treatment in patients afflicted with VHL disease. Investigators hypothesize that belzutifan might delay or obviate the need for serial surgeries that can burden such patients with substantial complications. (Jonasch, E. et al., N Engl J Med 2021; 385:2036-2046).

In studies of belzutifan for the treatment of RCC, the agent showed excellent in vitro potency and pharmacokinetic profiles and in vivo efficacy in mouse models, and has shown encouraging outcomes in patients with advanced RCC (Xu, Rui, et al., J. Med. Chem. 62:6876-6893 (2019). In a recent clinical study of patients having previously treated advanced clear cell RCC, the confirmed objective response rate was 25%, and the median progression-free survival was 14.5 months. (Choueri, T. K. et al., Nature Medicine vol. 27, 802-805 (2021)).

Due to its therapeutic effects in treating patients suffering from VHL disease and its potential in treating patients with RCC, efficient processes for preparing large scale quantities of belzutifan to support commercial supply and continuing clinical studies are needed. U.S. Patent Application Publication No. US2022/0881407 A1 discloses methods for preparing certain substituted indanes, including belzutifan. U.S. provisional Application No. 63/191,356, filed May 21, 2021 discloses crystalline forms of certain synthetic intermediates and certain processes for isolating such forms which are advantageous for the preparation of belzutifan.

While the aforementioned publication and application describe useful and scaleable processes for preparing belzutifan, further processes for preparing the compound are valuable, particularly those that minimize the use of hazardous reagents and transition metal catalysts. In particular, it would be desirable to explore new transformations that provide optically pure intermediates which minimize the environmental burden by using biocatalysts, such as enzymes. The present disclosure provides such enzymes and a process for making a useful intermediate to prepare belzutifan.

### SUMMARY OF THE INVENTION

The present disclosure relates to enzymes derived from the fungi Fusarium oxysporum c8D (hereinafter “FoPip4H enzymes”) capable of hydroxylating a substituted indanone to provide an optically pure alcohol. In some embodiments, the subject FoPip4H enzymes described herein are capable of converting 4-fluoro-7-(methylsulfonyl)-2,3-dihydro-1H-inden-1-one (II) to stereomerically pure (R)-4-fluoro-3-hydroxy-7-(methylsulfonyl)-2,3-dihydro-1H-inden-1-one (I), which is useful for the synthesis of belzutifan, 3-[[(1S,2S,3R)-2,3-difluoro-2,3-dihydro-1-hydroxy-7-(methylsulfonyl)-1H-inden-4-yl]oxy]-5-fluorobenzonitrile.

Additional embodiments describe processes for preparing the subject FoPip4H enzymes and processes for using the subject FoPip4H enzymes.

In one aspect, the present disclosure provides a polypeptide comprising an amino acid sequence having at least a 90% sequence identity to SEQ ID NO: 2. In some embodiments, the amino acid sequence has at least a 95% sequence identity to SEQ ID NO: 2. In certain embodiments, the amino acid sequence has at least a 98% sequence identity to SEQ ID NO: 2. In particular embodiments, the the amino acid sequence consists of SEQ ID NO: 2. In a specific embodiment, the polypeptide consists of SEQ ID NO:2.

In another aspect, the present disclosure provides a polynucleotide encoding any of the aforesaid polypeptides. In a particular embodiment, the polynucleotide comprises SEQ ID NO: 3.

In another aspect, the present disclosure provides an expression vector comprising any of the aforesaid polynucleotides, operably linked to one or more control sequences suitable for directing expression of the encoded polypeptide in a host cell. In some embodiments, the control sequence comprises a promoter. In certain embodiments, the promoter comprises an E. coli promoter.

In another aspect, the present disclosure provides a host cell comprising any of the aforesaid expression vectors. In certain embodiments, the host cell is E. coli.

In another aspect, the present disclosure provides a process for preparing a compound according to Formula (I)

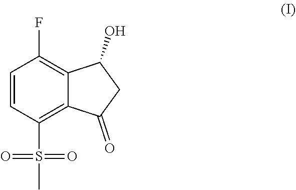 [SMILES: CS(=O)(=O)c1ccc(F)c2c1C(=O)C[C@H]2O]

in at least 60% enantiomeric excess (ee), the process comprising contacting the indanone according to Formula (II)

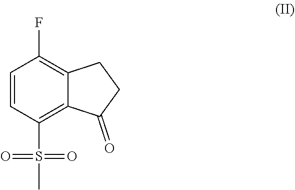

with α-ketoglutarate in the presence of the aforesaid polypeptides to provide the compound of Formula (I). In specific embodiments of the process, the compound according to Formula (I) is prepared in at least 80%, 90%, 95%, 98%, or 99% ee.

In some embodiments, the process further comprises a reductant selected from the group consisting of L-cysteine, ascorbic acid, dithiothreitol, D-cysteine, L-homocysteine, and D-cysteine ethyl ester.

In certain embodiments, the process is performed with a buffer selected from the group consisting of phosphate buffer, 2-morpholinoethanesulfonic acid, Bis Tris, PIPES, citrate, bicine, and TEOA.

In some embodiments, the process further comprises an iron salt selected from the group consisting of Mohr's salt ((NH₄)₂Fe(SO₄)₂6H₂O), and iron chloride.

Other embodiments, aspects and features of the present invention are either further described in or will be apparent from the ensuing description, examples and appended claims.

### DETAILED DESCRIPTION OF THE INVENTION

### Definitions

Certain technical and scientific terms are specifically defined below. Unless specifically defined elsewhere in this document, all other technical and scientific terms used herein have the meaning commonly understood by one of ordinary skill in the art to which this disclosure relates. That notwithstanding and except where stated otherwise, the following definitions apply throughout the specification and claims. Chemical names, common names, and chemical structures may be used interchangeably to describe the same structure

As used herein, and throughout this disclosure, the following terms, unless otherwise indicated, shall be understood to have the following meanings:

The present disclosure also embraces isotopically-labelled compounds that are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²³I, respectively.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Isotopic substitution at a site where epimerization occurs may slow or reduce the epimerization process and thereby retain the more active or efficacious form of the compound for a longer period of time. Isotopically labeled compounds, in particular those containing isotopes with longer half-lives (T_{1/2}>1 day), can generally be prepared by following procedures analogous to those disclosed in the Schemes and/or in the Examples herein below, by substituting an appropriate isotopically labeled reagent for a non-isotopically labeled reagent.

Compounds herein may contain one or more stereogenic centers and can occur as racemates, racemic mixtures, single enantiomers, diastereomeric mixtures, and individual diastereomers. Additional asymmetric centers may be present depending upon the nature of the various substituents on the molecule. Each such asymmetric center will independently produce two optical isomers, and all possible optical isomers and diastereomers in mixtures and as pure or partially purified compounds are included within the disclosure. Any formulas, structures, or names of compounds described herein that do not specify a particular stereochemistry are meant to encompass any and all existing isomers as described above and mixtures thereof in any proportion. When stereochemistry is specified, the disclosure is meant to encompass that particular isomer in pure form or as part of a mixture with other isomers in any proportion.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of chiral HPLC column.

All stereoisomers (for example, geometric isomers, optical isomers, and the like) of disclosed compounds (including those of the salts and solvates of compounds as well as the salts, solvates, and esters of prodrugs), such as those that may exist due to asymmetric carbons on various substituents, including enantiomeric forms (which may exist even in the absence of asymmetric carbons), rotameric forms, atropisomers, and diastereomeric forms, are contemplated within the scope of this disclosure. Individual stereoisomers of compounds may, for example, be substantially free of other isomers, or may be admixed, for example, as racemates or with all other, or other selected, stereoisomers. The chiral centers can have the S or R configuration as defined by the IUPAC 1974 Recommendations.

The present disclosure further includes compounds and synthetic intermediates in all their isolated forms. For example, the above-identified compounds are intended to encompass all forms of the compounds such as, any solvates, hydrates, stereoisomers, and tautomers thereof.

“Protein,” “polypeptide,” and “peptide” are used interchangeably herein to denote a polymer of at least two amino acids covalently linked by an amide bond, regardless of length or post-translational modification (e.g., glycosylation or phosphorylation, lipidation, myristoylation, ubiquitination, etc.). Included within this definition are D- and L-amino acids, and mixtures of D- and L-amino acids, as well as polymers comprising D- and L-amino acids, and mixtures of D- and L-amino acids. Proteins, polypeptides, and peptides may include a tag, such as a histidine tag, which should not be included when determining percentage of sequence identity.

“Amino acid” or “residue” as used in context of the polypeptides disclosed herein refers to the specific monomer at a sequence position. Amino acids are referred to herein by either their commonly known three-letter symbols or by the one-letter symbols recommended by IUPAC-IUB Biochemical Nomenclature Commission. Nucleotides, likewise, may be referred to by their commonly accepted single letter codes.

The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V).

The abbreviations used for the genetically encoding nucleosides are conventional and are as follows: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated.

“Derived from” as used herein in the context of enzymes, identifies the originating enzyme, and/or the gene encoding such enzyme, upon which the enzyme was based. For example, the FoPip4H enzyme of SEQ ID NO:2 was obtained by artificially evolving, over multiple generations the gene encoding the wild type (wt) FoPip4H enzyme of SEQ ID NO: 1. Thus, this evolved FoPip4H enzyme is “derived from” the FoPip4H of SEQ ID NO: 1.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides over a “comparison window” to identify and compare local regions of sequence similarity.

In some embodiments, a “reference sequence” can be based on a primary amino acid sequence, where the reference sequence is a sequence that can have one or more changes in the primary sequence. For instance, a reference sequence “based on SEQ ID NO: 1 having at the residue corresponding to X96 a leucine” refers to a reference sequence in which the corresponding residue at X96 in SEQ ID NO: 1 has been changed to a leucine.

“Hydrophilic amino acid or residue” refers to an amino acid or residue having a side chain exhibiting a hydrophobicity of less than zero according to the normalized consensus hydrophobicity scale of Eisenberg et al., 1984, J. Mol. Biol. 179:125-142. Genetically encoded hydrophilic amino acids include L-Thr (T), L-Ser(S), L-His (H), L-Glu (E), L-Asn (N), L-Gln (Q), L-Asp (D), L-Lys (K), and L-Arg (R).

“Acidic amino acid or residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

“Basic amino acid or residue” refers to a hydrophilic amino acid or residue having a side chain exhibiting a pKa value of greater than about 6 when the amino acid is included in a peptide or polypeptide. Basic amino acids typically have positively charged side chains at physiological pH due to association with hydronium ion. Genetically encoded basic amino acids include L-Arg (R) and L-Lys (K).

“Polar amino acid or residue” refers to a hydrophilic amino acid or residue having a side chain that is uncharged at physiological pH, but which has at least one bond in which the pair of electrons shared in common by two atoms is held more closely by one of the atoms. Genetically encoded polar amino acids include L-Asn (N), L-Gln (Q), L-Ser(S), and L-Thr (T).

“Hydrophobic amino acid or residue” refers to an amino acid or residue having a side chain exhibiting a hydrophobicity of greater than zero according to the normalized consensus hydrophobicity scale of Eisenberg et al., 1984, J. Mol. Biol. 179:125-142. Genetically encoded hydrophobic amino acids include L-Pro (P), L-Ile (I), L-Phe (F), L-Val (V), L-Leu (L), L-Trp (W), L-Met (M), L-Ala (A), and L-Tyr (Y).

“Aromatic amino acid or residue” refers to a hydrophilic or hydrophobic amino acid or residue having a side chain that includes at least one aromatic or heteroaromatic ring. Genetically encoded aromatic amino acids include L-Phe (F), L-Tyr (Y), L-His (H), and L-Trp (W). L-His (H) histidine is also classified herein as a hydrophilic residue or as a constrained residue.

As used herein, “constrained amino acid or residue” refers to an amino acid or residue that has a constrained geometry. Herein, constrained residues include L-Pro (P) and L-His (H). Histidine has a constrained geometry because it has a relatively small imidazole ring. Proline has a constrained geometry because it also has a five-membered ring.

“Non-polar amino acid or residue” refers to a hydrophobic amino acid or residue that has a side chain that is uncharged at physiological pH and that has bonds in which the pair of electrons shared in common by two atoms is generally held equally by each of the two atoms (i.e., the side chain is not polar). Genetically encoded non-polar amino acids include L-Gly (G), L-Leu (L), L-Val (V), L-Ile (I), L-Met (M), and L-Ala (A).

As used herein, “aliphatic amino acid or residue” refers to a hydrophobic amino acid or residue having an aliphatic hydrocarbon side chain. Genetically encoded aliphatic amino acids include L-Ala (A), L-Val (V), L-Leu (L), and L-Ile (I).

The ability of L-Cys (C) (and other amino acids with-SH containing side chains) to exist in a peptide in either the reduced free-SH or oxidized disulfide-bridged form affects whether L-Cys (C) contributes net hydrophobic or hydrophilic character to a peptide. While L-Cys (C) exhibits a hydrophobicity of 0.29 according to the normalized consensus scale of Eisenberg (Eisenberg et al., 1984, supra), it is to be understood that for purposes of the present disclosure, L-Cys (C) is categorized into its own unique group. It is noted that cysteine (or “L-Cys” or “[C]”) is unusual in that it can form disulfide bridges with other L-Cys (C) amino acids or other sulfanyl-or sulfhydryl-containing amino acids. The “cysteine-like residues” include cysteine and other amino acids that contain sulfhydryl moieties that are available for formation of disulfide bridges.

As used herein, “small amino acid or residue” refers to an amino acid or residue having a side chain that is composed of a total three or fewer carbon and/or heteroatoms (excluding the α-carbon and hydrogens). The small amino acids or residues may be further categorized as aliphatic, non-polar, polar or acidic small amino acids or residues, in accordance with the above definitions. Genetically-encoded small amino acids include L-Ala (A), L-Val (V), L-Cys (C), L-Asn (N), L-Ser(S), L-Thr (T), and L-Asp (D).

As used herein, “polynucleotide” and “nucleic acid” refer to two or more nucleotides that are covalently linked together. The polynucleotide may be wholly comprised of ribonucleotides (i.e., RNA), wholly comprised of 2′ deoxyribonucleotides (i.e., DNA), or comprised of mixtures of ribo- and 2′ deoxyribonucleotides. While the nucleosides will typically be linked together via standard phosphodiester linkages, the polynucleotides may include one or more non-standard linkages. The polynucleotide may be single-stranded or double-stranded, or the polynucleotide may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. In some embodiments, such modified or synthetic nucleobases are nucleobases encoding amino acid sequences.

“Hydroxyl-containing amino acid or residue” refers to an amino acid containing a hydroxyl (—OH) moiety. Genetically-encoded hydroxyl-containing amino acids include L-Ser(S) L-Thr (T), and L-Tyr (Y).

As used herein, “conservative amino acid substitution” refers to a substitution of a residue with a different residue having a similar side chain, and thus typically involves substitution of the amino acid in the polypeptide with amino acids within the same or similar defined class of amino acids. By way of example and not limitation, in some embodiments, an amino acid with an aliphatic side chain is substituted with another aliphatic amino acid (e.g., alanine, valine, leucine, and isoleucine); an amino acid with an hydroxyl side chain is substituted with another amino acid with an hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basic side chain (e.g., lysine and arginine); an amino acid with an acidic side chain is substituted with another amino acid with an acidic side chain (e.g., aspartic acid and glutamic acid); and/or a hydrophobic or hydrophilic amino acid is replaced with another hydrophobic or hydrophilic amino acid, respectively.

As used herein, “non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, “deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an evolved enzyme. Deletions can be directed to the internal portions and/or terminal portions of the polypeptide. In various embodiments, the deletion can comprise a continuous segment or can be discontinuous. Deletions are typically indicated by “-” in amino acid sequences.

As used herein, “insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

The term “amino acid substitution set” or “substitution set” refers to a group of amino acid substitutions in a polypeptide sequence, as compared to a reference sequence. A substitution set can have 1, 2, 3, 4, 5, 6, 7, 8, 9, 10, 11, 12, 13, 14, 15, or more amino acid substitutions.

A “functional fragment” and “biologically active fragment” are used interchangeably herein to refer to a polypeptide that has an amino-terminal and/or carboxy-terminal deletion(s) and/or internal deletions, but where the remaining amino acid sequence is identical to the corresponding positions in the sequence to which it is being compared and that retains substantially all of the activity of the full-length polypeptide.

As used herein, “isolated polypeptide” refers to a polypeptide that is substantially separated from other contaminants that naturally accompany it (e.g., protein, lipids, and polynucleotides). The term embraces polypeptides that have been removed or purified from their naturally occurring environment or expression system (e.g., within a host cell or via in vitro synthesis). The recombinant polypeptides may be present within a cell, present in the cellular medium, or prepared in various forms, such as lysates or isolated preparations. As such, in some embodiments, the recombinant polypeptides can be an isolated polypeptide.

As used herein, “substantially pure polypeptide” or “purified protein” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. However, in some embodiments, an enzyme comprising composition comprises enzymes that are less than 50% pure (e.g., about 10%, about 20%, about 30%, about 40%, or about 50%). Generally, a substantially pure enzyme or polypeptide composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant polypeptides are substantially pure polypeptide compositions.

“Improved enzyme property” refers to an enzyme that exhibits an improvement in any enzyme property as compared to a reference enzyme. For the enzymes described herein, the comparison is generally made to the wild-type enzyme, although in some embodiments, the reference enzyme can be another improved enzyme. Enzyme properties for which improvement is desirable include, but are not limited to, enzymatic activity (which can be expressed in terms of percent conversion of the substrate), thermal stability, pH activity profile, cofactor requirements, refractoriness to inhibitors (e.g., product inhibition), stereospecificity, and stereoselectivity (including enantioselectivity).

“Increased enzymatic activity” refers to an improved property of the enzymes, which can be represented by an increase in specific activity (e.g., product produced/time/weight protein) or an increase in percent conversion of the substrate to the product (e.g., percent conversion of starting amount of substrate to product in a specified time period using a specified amount of enzyme) as compared to the reference enzyme. Exemplary methods to determine enzyme activity are provided in the Examples. Any property relating to enzyme activity may be affected, including the classical enzyme properties of Kₘ, Vₘₐₓ, or k_{cat}, changes of which can lead to increased enzymatic activity. Improvements in enzyme activity can be from about 1.5 times the enzymatic activity of the corresponding wild-type enzyme, to as much as 2 times. 5 times, 10 times, 20 times, 25 times, 50 times, 75 times, 100 times, 150 times, 200 times, 500 times, 1000 times, 3000 times, 5000 times, 7000 times or more enzymatic activity than the naturally occurring enzyme or another enzyme from which the polypeptides were derived. In specific embodiments, the enzyme exhibits improved enzymatic activity in the range of 150 to 3000 times, 3000 to 7000 times, or more than 7000 times greater than that of the parent enzyme. It is understood by the skilled artisan that the activity of any enzyme is diffusion limited such that the catalytic turnover rate cannot exceed the diffusion rate of the substrate, including any required cofactors. The theoretical maximum of the diffusion limit, or k_{cat}/Kₘ, is generally about 10⁸ to 10⁹ (M⁻¹s⁻¹). Hence, any improvements in the enzyme activity will have an upper limit related to the diffusion rate of the substrates acted on by the enzyme. Enzyme activity can be measured by any one of standard assays used for measuring kinase activity, or via a coupled assay with an nucleoside phosphorylase enzyme which is capable of catalyzing reaction between the polypeptide product and a nucleoside base to afford a nucleoside, or by any of the traditional methods for assaying chemical reactions, including but not limited to HPLC, HPLC-MS, UPLC, UPLC-MS, TLC, and NMR. Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

As used herein, a “vector” is a DNA construct for introducing a DNA sequence into a cell. In some embodiments, the vector is an expression vector that is operably linked to a suitable control sequence capable of effecting the expression in a suitable host of the polypeptide encoded in the DNA sequence. In some embodiments, an “expression vector” has a promoter sequence operably linked to the DNA sequence (e.g., transgene) to drive expression in a host cell, and in some embodiments, also comprises a transcription terminator sequence.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

As used herein, an amino acid or nucleotide sequence (e.g., a promoter sequence, signal peptide, terminator sequence, etc.) is “heterologous” to another sequence with which it is operably linked if the two sequences are not associated in nature. For example, a “heterologous polynucleotide” is any polynucleotide that is introduced into a host cell by laboratory techniques, and the term includes polynucleotides that are removed from a host cell, subjected to laboratory manipulation, and then reintroduced into a host cell.

As used herein, the terms “host cell” and “host strain” refer to suitable hosts for expression vectors comprising DNA provided herein (e.g., the polynucleotides encoding the variants). In some embodiments, the host cells are prokaryotic or eukaryotic cells that have been transformed or transfected with vectors constructed using recombinant DNA techniques as known in the art.

The term “analogue” means a polypeptide having more than 70% sequence identity but less than 100% sequence identity (e.g., more than 75%, 78%, 80%, 83%, 85%, 88%, 90%, 91%, 92%, 93%, 94%, 95%, 96%, 97%, 98%, or 99% sequence identity) with a reference polypeptide. In some embodiments, “analogues” means polypeptides that contain one or more non-naturally occurring amino acid residues including, but not limited, to homoarginine, ornithine and norvaline, as well as naturally occurring amino acids. In some embodiments, analogues also include one or more D-amino acid residues and non-peptide linkages between two or more amino acid residues.

As used herein, “EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

As used herein, “ATCC” refers to the American Type Culture Collection whose biorepository collection includes genes and strains.

As used herein, “NCBI” refers to National Center for Biological Information and the sequence databases provided therein.

“Coding sequence” refers to that portion of a nucleic acid (e.g., a gene) that encodes an amino acid sequence of a protein.

“Naturally occurring” or “wild-type” refers to a form found in nature. For example, a naturally occurring or wild-type polypeptide or polynucleotide sequence is a sequence present in an organism that can be isolated from a source in nature and that has not been intentionally modified by human manipulation, with the sole exception that wild-type polypeptide or polynucleotide sequences as identified herein may include a tag, such as a histidine tag, which should not be included when determining percentage of sequence identity. Herein, “wild-type” polypeptide or polynucleotide sequences may be denoted “WT”.

“Recombinant” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percentage of sequence identity,” “percent identity,” and “percent identical” are used herein to refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see e.g., Altschul et al., 1990, J. Mol. Biol. 215:403-410; and Altschul et al., 1977, Nucleic Acids Res. 3389-3402). Software for performing BLAST analyses is publicly available through the National Center for Biotechnology Information website.

Briefly, the BLAST analyses involve first identifying high scoring sequence pairs (HSPs) by identifying short words of length W in the query sequence, which either match or satisfy some positive-valued threshold score T when aligned with a word of the same length in a database sequence. Tis referred to as, the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff and Henikoff, 1989, PROC. NATL. ACAD. SCI. USA 89:10915).

Numerous other algorithms are available that function similarly to BLAST in providing percent identity for two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison WI), using default parameters provided.

“Substantial identity” refers to a polynucleotide or polypeptide sequence that has at least 80 percent sequence identity, preferably at least 85 percent sequence identity, more preferably at least 89 percent sequence identity, more preferably at least 95 percent sequence identity, and even more preferably at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, more preferably at least 95 percent sequence identity or more (e.g., 99 percent sequence identity). Preferably, residue positions which are not identical differ by conservative amino acid substitutions.

“Corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Stereoselectivity” refers to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. Itis commonly alternatively reported in the art (typically as a percentage) as the enantiomeric excess (EE) calculated therefrom according to the formula [major enantiomer-minor enantiomer]/[major enantiomer+minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (DE). Enantiomeric excess and diastereomeric excess are types of stereomeric excess.

“Highly stereoselective” refers to a chemical or enzymatic reaction that is capable of converting a substrate to its corresponding product with at least about 85% stereoisomeric excess.

“Chemoselectivity” refers to the preferential formation in a chemical or enzymatic reaction of one product over another.

“Conversion” refers to the enzymatic transformation of a substrate to the corresponding product. “Percent conversion” refers to the percent of the substrate that is converted to the product within a period of time under specified conditions. Thus, for example, the “enzymatic activity” or “activity” of a polypeptide can be expressed as “percent conversion” of the substrate to the product.

“Chiral alcohol” refers to amines of general formula R¹—CH(OH)—R² wherein R¹ and R² are nonidentical and is employed herein in its broadest sense, including a wide variety of aliphatic and alicyclic compounds of different, and mixed, functional types, characterized by the presence of a primary hydroxyl group bound to a secondary carbon atom which, in addition to a hydrogen atom, carries either (i) a divalent group forming a chiral cyclic structure, or (ii) two substituents (other than hydrogen) differing from each other in structure or chirality. Divalent groups forming a chiral cyclic structure include, for example, 2-methylbutane-1,4-diyl, pentane-1,4-diyl, hexane-1,4-diyl, hexane-1,5-diyl, 2-methylpentane-1,5-diyl. The two different substituents on the secondary carbon atom (R¹ and R² above) also can vary widely and include alkyl, aralkyl, aryl, halo, hydroxy, lower alkyl, lower alkoxy, lower alkylthio, cycloalkyl, carboxy, carboalkoxy, carbamoyl, mono- and di-(lower alkyl) substituted carbamoyl, trifluoromethyl, phenyl, nitro, amino, mono- and di-(lower alkyl) substituted amino, alkylsulfonyl, arylsulfonyl, alkylcarboxamido, arylcarboxamido, etc., as well as alkyl, aralkyl, or aryl substituted by the foregoing.

Immobilized enzyme preparations have a number of recognized advantages. They can confer shelf life to enzyme preparations, they can improve reaction stability, they can enable stability in organic solvents, they can aid in protein removal from reaction streams, as examples. “Stable” refers to the ability of the immobilized enzymes to retain their structural conformation and/or their activity in a solvent system that contains organic solvents. Stable immobilized enzymes lose less than 10% activity per hour in a solvent system that contains organic solvents. Stable immobilized enzymes lose less than 9% activity per hour in a solvent system that contains organic solvents. Preferably, the stable immobilized enzymes lose less than 8% activity per hour in a solvent system that contains organic solvents. Preferably, the stable immobilized enzymes lose less than 7% activity per hour in a solvent system that contains organic solvents. Preferably, the stable immobilized enzymes lose less than 6% activity per hour in a solvent system that contains organic solvents. Preferably, the stable immobilized enzymes lose less than 5% activity per hour in a solvent system that contains organic solvents. Preferably, the stable immobilized enzymes less than 4% activity per hour in a solvent system that contains organic solvents. Preferably, the stable immobilized enzymes lose less than 3% activity per hour in a solvent system that contains organic solvents. Preferably, the stable immobilized enzymes lose less than 2% activity per hour in a solvent system that contains organic solvents. Preferably, the stable immobilized enzymes lose less than 1% activity per hour in a solvent system that contains organic solvents.

“Thermostable” refers to a polypeptide that maintains similar activity (more than 60% to 80%, for example) after exposure to elevated temperatures (e.g., 40° C. to 80° C.) for a period of time (e.g., 0.5h to 24h) compared to the untreated enzyme.

“Solvent stable” refers to a polypeptide that maintains similar activity (more than e.g., 60% to 80%) after exposure to varying concentrations (e.g., 5% to 99%) of solvent (isopropyl alcohol, tetrahydrofuran, 2-methyltetrahydrofuran, acetone, toluene, butylacetate, methyl tert-butylether, etc.) for a period of time (e.g., 0.5h to 24h) compared to the untreated enzyme.

“pH stable” refers to a polypeptide that maintains similar activity (more than e.g., 60% to 80%) after exposure to high or low pH (e.g., 4.5 to 6 or 8 to 12) for a period of time (e.g., 0.5h to 24h) compared to the untreated enzyme.

“Thermo- and solvent stable” refers to a polypeptide that is both thermostable and solvent stable.

As used herein, the terms “biocatalysis,” “biocatalytic,” “biotransformation,” and “biosynthesis” refer to the use of enzymes to perform chemical reactions on organic compounds.

The term “effective amount” means an amount sufficient to produce the desired result. One of general skill in the art may determine what the effective amount by using routine experimentation.

The terms “isolated” and “purified” are used to refer to a molecule (e.g., an isolated nucleic acid, polypeptide, etc.) or other component that is removed from at least one other component with which it is naturally associated. The term “purified” does not require absolute purity, rather it is intended as a relative definition.

“Control sequence” is defined herein to include all components, which are necessary or advantageous for the expression of a polynucleotide and/or polypeptide of interest. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with a polynucleotide of interest, e.g., the coding region of the nucleic acid sequence encoding a polypeptide.

“Operably linked” is defined herein as a configuration in which a control sequence is appropriately placed at a position relative to a polynucleotide sequence (i.e., in a functional relationship) such that the control sequence directs the expression of the polynucleotide and/or a polypeptide encoded by the polynucleotide.

“Promoter sequence” is a nucleic acid sequence that is recognized by a host cell for expression of a polynucleotide. The control sequence may comprise an appropriate promoter sequence. The promoter sequence contains transcriptional control sequences, which mediate the expression of the polynucleotide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Co-substrate” of a FoPip4H enzyme refers to α-ketoglutarate and co-substrate analogs that can replace α-ketoglutarate ketoglutarate in hydroxylation of indanone substrate analogs. Co-substrate analogs include, by way of example and not limitation, 2-oxoadipate (see, e.g., Majamaa et al., 1985, Biochem. J. 229:127-133).

“FoPip4H enzyme” or “FoPip4H polypeptides” refers to a polypeptide having an enzymatic capability of oxidizing a cyclic substrate to provide an alcohol-substituted cyclic structure. More specifically, the FoPip4H polypeptides disclosed are capable of stereoselectively hydroxylating the substituted indanone of Formula (II), to the substituted 3-hydroxyindanone of Formula (I), (shown above). FoPip4H enzyme as used herein includes naturally occurring (wild type) hydroxylases as well as non-naturally occurring engineered polypeptides generated by human manipulation.

Exemplary methods and materials are described herein, although methods and materials similar or equivalent to those described herein can also be used in the practice or testing of the present disclosure. The materials, methods, and examples are illustrative only and not intended to be limiting.

### ABBREVIATIONS

ACN, MeCN Acetonitrile
Bicine [Bis(2-hydroxyethyl)amino]acetic acid
Bis Tris 2-Bis(2-hydroxyethyl)amino-2-(hydroxymethyl)-1,3-propanediol
DMSO Dimethyl sulfoxide
g Grams
h Hour
Hz Hertz
IPA Isopropyl alcohol
IPTG Isopropyl β-D-1-thiogalactopyranoside
J NMR Coupling constant
L Liter
M Molar, moles per liter
mg Milligrams
min Minute(s)
mL, ml Milliliters
mm Millimeter
mM Millimole per liter
nm Nanometer
NaCl Sodium chloride
NADP Nicotinamide adenine dinucleotide phosphate
OD₆₀₀ Optical density at 600 nm wavelength
PIPES Piperazine-N,N-bis(2-ethanesulfonic acid
PTFE Polytetrafluoroethylene
RH Relative humidity
RPM, rpm Revolutions per minute
RT Room temperature, approximately 25° C.
SFC Supercritical Fluid Chromatography
T₅₀ Temperature at which 50% of the enzymatic activity is lost
TEOA triethanolamine
TFA Trifluoroacetic acid
THF Tetrahydrofuran
UPLC Ultra-Performance Liquid Chromatography
vol Volumes
v/v Volume per volume
μg, μg Micrograms
μL, μl, μL, μl Microliters

### FoPip4H Enzymes

This disclosure relates to FoPip4H enzymes capable of hydroxylating a substituted indanone to provide an optically pure alcohol. In embodiments, the FoPip4H enzymes are capable of the following conversion:

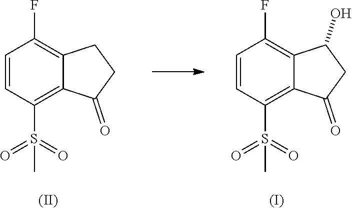

In certain embodiments, the FoPip4H enzymes described herein have an amino acid sequence that has one or more amino acid differences as compared to a reference amino acid sequence of a wild type FoPip4H that result in an improved property of the enzyme for the defined indanone substrate.

The FoPip4H enzymes described herein are the product of directed evolution from the wild type FoPip4H c8D (SEQ ID NO:1), which was identified by screening a panel of literature referenced enzymes and which has the amino acid sequence as set forth below (including an added histidine tag):

(SEQ ID NO: 1)
MGSHHHHHHHHGSAALNADTLDMSLFFGTPSQKQDFCDSLLRLLKKRGG
VKLINHPIPSTSIHELFAQTKRFFNLPLETKMLAKHPPQANPNRGYSFV
GQENVANISGYEKGLGPLKTRDIKETVDFGSANDELVDNLWVPEEELPG
FRSFMEGFYELAFKTEMQLLEALAIALGVSPDHLKSLHNRAENEFRILH
YPAIPASELADGTATRIAEHTDFGTITMLFQDSVGGLQVEDQENLGTFN
NVESASPTDIILNIGDSLQRLTNDTFKAACHRVTYPPSIKAGDGEQVIP
ERYSIAYFAKPNRSASLFPLKEFIEEGVPCKYEDVTAWEWNNRRIEKLF
SAEAKA.

In embodiments, the FoPip4H enzymes of the disclosure may demonstrate improvements relative the FoPip4H enzyme of SEQ ID NO: 1, such as increases in enzyme activity, stereoselectivity, stereospecificity, thermostability, solvent stability, reduced product inhibition, or reduced overoxidation.

In some embodiments, the FoPip4H enzymes of the disclosure may demonstrate improvements in the rate of enzymatic activity, i.e., the rate of converting an indanone substrate to the product. In some embodiments, the FoPip4H polypeptides are capable of converting the substrate to the product at a rate that is at least 1.5-times, 2-times, 3-times, 4-times, 5-times, 10-times, 25-times, 50-times, 100-times, 150-times, 200-times, 400-times, 1000-times, 3000-times, 5000-times, 7000-times or more than 7000-times the rate exhibited by the enzyme of SEQ ID NO: 1.

In some embodiments, such FoPip4H polypeptides are also capable of converting the indanone substrate to the product with a percent enantiomeric excess of at least 60%. In some embodiments, such FoPip4H polypeptides are also capable of converting the substrate to the product with a percent enantiomeric excess of at least 90%. In some embodiments, such FoPip4H polypeptides are also capable of converting the substrate to the product with a percent enantiomeric excess of at least about 99%.

In some embodiments, the FoPip4H polypeptide is highly enantioselective, wherein the polypeptide can reduce the substrate to the product in greater than about 99%, 99.1%, 99.2%, 99.3%, 99.4%, 99.5%, 99.6%, 99.7%, 99.8%, or 99.9% enantiomeric excess.

In some embodiments, an improved FoPip4H polypeptide of the disclosure is a polypeptide that comprises an amino acid sequence having at least 90% sequence identity to SEQ ID NO: 2 which has the amino acid sequence as set forth below:

(SEQ ID NO: 2)
MGSHHHHHHHHGSAALNADTLDMSLFFGTPSQKQDFCDSLLRLLKARGV
VKLINHPIPSESIHELFAQTKRFFNLPLETKMLAKHPEQALPARGYAFV
GQENVANISGYEKGLPPLKTRDIKETVDFGSANDEKYDNLWVPEEELPG
FRSFMEGFYELAFKTEMQILEALAIALGVSPDHLKSLHNRAHSELRILH
YPAIPASELADGTATRIAEHTDFGSITMLFQDGVGGLQVEDQENLGTFN
NVESASPTDIILNIGDSLQRLTNDTFKAACHRVTWPPSIKDGDGSEVIP
ERYSVAYFVKPNRSASLFPLKEFIEEGVPPKYEDLTFEEWNNRRIEKLF
SAEAKA

In some embodiments, an improved FoPip4H polypeptide of the disclosure is based on the sequence formulas of SEQ ID NO: 2 and can comprise an amino acid sequence that is at least 85%, 86%, 87%, 88%, 89%, 90%, 91%, 92%, 93%, 94%, 95%, 96%, 97%, 98%, or 99% identical to the reference sequence of SEQ ID NO: 2.

These differences between these variants and SEQ ID NO:2 can be amino acid insertions, deletions, substitutions, or any combinations of such changes. In some embodiments, the amino acid sequence differences can comprise non-conservative, conservative, as well as a combination of non-conservative and conservative amino acid substitutions.

In particular embodiments, an improved FoPip4H polypeptide of the disclosure wherein the amino acid sequence consists of SEQ ID NO: 2. In specific embodiments, the improved FoPip4H polypeptide of the disclosure consists of SEQ ID NO: 2.

Additional embodiments provide host cells comprising the polynucleotides and/or expression vectors described herein. The host cells may be E. coli, or they may be a different organism, such as L. brevis. The host cells can be used for the expression and isolation of the FoPip4H enzymes described herein, or, alternatively, they can be used directly for the conversion of the substrate to the stereoisomeric product.

Whether carrying out the method with whole cells, cell extracts or purified FoPip4H enzymes, a single FoPip4H enzyme may be used or, alternatively, mixtures of two or more FoPip4H enzymes may beused.

Table 1 below provides a list of the FoPip4H polypeptides (identified by the SEQ ID NOs disclosed herein) and their associated properties. The sequences below are based on the FoPip4H sequence of SEQ ID NO: 1, unless otherwise specified. In table below, each row lists a SEQ ID NO. The column listing the number of mutations (i.e., residue changes) refers to the number of amino acid substitutions as compared to the FoPip4H sequence of SEQ ID NO: 1.

In the thermostability column of the table: + indicates T₅₀<25° C.; ++ indicates T₅₀>25° C.<45° C.; +++ indicates T₅₀>45° C. In the conversion column of the table: + indicates <10% conversion of substrate to product; ++ indicates 10%-60% conversion; +++ indicates >60% conversion. The conversion analysis was performed based on the assumption of 25 wt. % enzyme and 10 g/L of the substrate. In the selectivity column of the table: + indicates <60% enantiomeric excess (ee); ++ indicates 60%-90% ee; +++ indicates >90% ee. In the overoxidation column of the table: +++ indicates >15% overoxidation; ++ indicates 5%-15% overoxidation; + indicates <5% overoxidation.

TABLE 1
Activity of FoPip4H Enzymes

| | | No. of Changes | | | | |
|---|---|---|---|---|---|---|
| SEQ | Residue Changes Relative to | Relative to | Thermo- | % | | % Over- |
| ID NO: | SEQ ID NO: 1 | SEQ ID NO: 1 | stability | Conversion | Selectivity | oxidation |
| 1 | — | 0 | + | + | + | |
| 4 | S96L | 1 | + | + | ++ | |
| 5 | N92A; S96L; F192L; A303V | 4 | ++ | ++ | +++ | |
| 6 | N90L; N92A; S96L; V135Y; F192L; | 6 | ++ | ++ | +++ | |
| | A303V | | | | | |
| 7 | N90L; N92A; S96L; V135Y; F192L; | 7 | ++ | ++ | +++ | |
| | A303V; W332E | | | | | |
| 8 | K46A; N90L; N92A; S96L; G114S; | 10 | | +++ | +++ | |
| | V135Y; F192L; I299V; A303V; W332E | | | | | |
| 9 | K46A; N90L; N92A; S96L; G114S; | 11 | ++ | +++ | +++ | +++ |
| | V135Y; E189H; F192L; I299V; A303V; | | | | | |
| | W332E | | | | | |
| 10 | K46A; A89P; N90L; N92A; S96L; | 13 | | +++ | +++ | +++ |
| | G114S; V135Y; E189H; F192L; T221S; | | | | | |
| | I299V; A303V; W332E | | | | | |
| 11 | K46A; N90L; N92A; G114S; V135Y; | 11 | ++ | +++ | +++ | +++ |
| | E189H; F192L; T221S; I299V; | | | | | |
| | A303V; W332E | | | | | |
| 12 | K46A; T60E; N90L; N92A; G114S; | 12 | +++ | +++ | +++ | +++ |
| | V135Y; E189H; F192L; T221S; I299V; | | | | | |
| | A303V; W332E | | | | | |
| 13 | K46A; T60E; N90L; N92A; S96A; | 13 | +++ | +++ | +++ | +++ |
| | G114S; V135Y; E189H; F192L; T221S; | | | | | |
| | I299V; A303V; W332E | | | | | |
| 14 | K46A; T60E; N90L; N92A; S96A; | 16 | | +++ | +++ | ++ |
| | G114P; L134K; V135Y; E189H; F192L; | | | | | |
| | T221S; S229G; A286D; I299V; A303V; | | | | | |
| | W332E | | | | | |
| 15 | K46A; G49V; T60E; N90L; N92A; | 21 | | +++ | +++ | ++ |
| | S96A; Y109W; G114P; L134K; V135Y; | | | | | |
| | E189H; F192L; T221S; S229G; Y280W; | | | | | |
| | A286D; E290T; I299V; A303V; | | | | | |
| | V329L; W332E | | | | | |
| 16 | K46A; G49V; T60E; P87E; N90L; | 24 | ++ | +++ | +++ | + |
| | N92A; S96A; G114P; L134K; V135Y; | | | | | |
| | E189H; N190S; F192L; T221S; S229G; | | | | | |
| | Y280W; A286D; E290S; Q291E; | | | | | |
| | I299V; A303V; C324P; V329L; W332E | | | | | |
| 17 | K46A; G49V; T60E; P87E; N90L; | 25 | +++ | +++ | +++ | + |
| | N92A; S96A; G114P; L134K; V135Y; | | | | | |
| | L166I; E189H; N190S; F192L; T221S; | | | | | |
| | S229G; Y280W; A286D; E290S; | | | | | |
| | Q291E; I299V; A303V; C324P; V329L; | | | | | |
| | W332E | | | | | |
| 2 | K46A; G49V; T60E; P87E; N90L; | 26 | +++ | +++ | +++ | + |
| | N92A; S96A; G114P; L134K; V135Y; | | | | | |
| | L166I; E189H; N190S; F192L; T221S; | | | | | |
| | S229G; Y280W; A286D; E290S; | | | | | |
| | Q291E; I299V; A303V; C324P; V329L; | | | | | |
| | A331F; W332E | | | | | |
| 18 | K46A; R47K; G49V; S59A; T60E; | 30 | +++ | +++ | +++ | + |
| | H63K; P87E; N90L; N92A; S96A; G114P; | | | | | |
| | L134K; V135Y; L166I; R187G; | | | | | |
| | E189H; N190S; F192L; T221S; S229G; | | | | | |
| | Y280W; A286D; E290S; Q291E; | | | | | |
| | I299V; A303V; C324P; V329L; A331F; | | | | | |
| | W332E | | | | | |
| 19 | K46A; R47K; G49V; S59A; T60E; | 31 | +++ | +++ | +++ | + |
| | H63K; P87E; N90L; N92A; S96A; G114P; | | | | | |
| | L134K; V135Y; L166I; R187S; | | | | | |
| | E189H; N190S; F192L; T221S; S229G; | | | | | |
| | K272R; Y280W; A286D; E290S; | | | | | |
| | Q291E; I299V; A303V; C324P; V329L; | | | | | |
| | A331F; W332E | | | | | |

### Polynucleotides Encoding FoPip4H Enzymes

In another aspect, the present disclosure provides polynucleotides encoding the FoPip4H polypeptides disclosed herein. The polynucleotides may be operatively linked to one or more heterologous regulatory sequences that control gene expression to create a recombinant polynucleotide capable of expressing the polypeptide. Expression constructs containing a heterologous polynucleotide encoding the FoPip4H can be introduced into appropriate host cells to express the corresponding FoPip4H polypeptide.

Because of the knowledge of the codons corresponding to the various amino acids, availability of a protein sequence provides a description of all the polynucleotides capable of encoding the subject. The degeneracy of the genetic code, where the same amino acids are encoded by alternative or synonymous codons allows an extremely large number of nucleic acids to be made, all of which encode the improved FoPip4H enzymes disclosed herein. Thus, having identified a particular amino acid sequence, those skilled in the art could make any number of different nucleic acids by simply modifying the sequence of one or more codons in a way that does not change the amino acid sequence of the protein. In this regard, the present disclosure specifically contemplates each and every possible variation of polynucleotides that could be made by selecting combinations based on the possible codon choices, and all such variations are to be considered specifically disclosed for any polypeptide disclosed herein.

In various embodiments, the codons are preferably selected to fit the host cell in which the protein is being produced. For example, preferred codons used in bacteria are used to express the gene in bacteria; preferred codons used in yeast are used for expression in yeast; and preferred codons used in mammals are used for expression in mammalian cells. By way of example, the polynucleotide of SEQ ID NO: 3 has been codon optimized for expression in E. coli.

In certain embodiments, all codons need not be replaced to optimize the codon usage of the FoPip4H enzymes since the natural sequence will comprise preferred codons and because use of preferred codons may not be required for all amino acid residues. Consequently, codon optimized polynucleotides encoding the FoPip4H enzymes may contain preferred codons at about 40%, 50%, 60%, 70%, 80%, or greater than 90% of codon positions of the full length coding region.

In various embodiments, an isolated polynucleotide encoding an improved FoPip4H polypeptide may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, Molecular Cloning: A Laboratory Manual, 3^{rd} Ed., Cold Spring Harbor Laboratory Press; and Current Protocols in Molecular Biology, Ausubel. F. ed., Greene Pub. Associates, 1998, updates to 2006.

In some embodiments, an isolated polynucleotide encoding any of the FoPip4H polypeptides herein is manipulated in a variety of ways to facilitate expression of the FoPip4H polypeptide. In some embodiments, the polynucleotides encoding the FoPip4H polypeptides comprise expression vectors where one or more control sequences is present to regulate the expression of the FoPip4H polynucleotides and/or polypeptides. Manipulation of the isolated polynucleotide prior to its insertion into a vector may be desirable or necessary depending on the expression vector utilized. Techniques for modifying polynucleotides and nucleic acid sequences utilizing recombinant DNA methods are well known in the art. In some embodiments, the control sequences include among others, promoters, leader sequences, polyadenylation sequences, propeptide sequences, signal peptide sequences, and transcription terminators. In some embodiments, suitable promoters are selected based on the host cells selection. For bacterial host cells, suitable promoters for directing transcription of the nucleic acid constructs of the present disclosure, include, but are not limited to, promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See e.g., Villa-Kamaroff et al., Proc. Natl Acad. Sci. USA 75:3727-3731) [1978]), as well as the tac promoter (See e.g., DeBoer et al., Proc. Natl Acad. Sci. USA 80:21-25 [1983]). Exemplary promoters for filamentous fungal host cells, include, but are not limited to, promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillusniger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast 8:423-488 [1992]).

In some embodiments, the control sequence is also a suitable transcription terminator sequence (i.e., a sequence recognized by a host cell to terminate transcription). In some embodiments, the terminator sequence is operably linked to the 3′ terminus of the nucleic acid sequence encoding the enzyme polypeptide. Any suitable terminator that is functional in the host cell of choice finds use in the present invention. Exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra).

In some embodiments, the control sequence is also a suitable leader sequence (i.e., a non-translated region of an mRNA that is important for translation by the host cell). In some embodiments, the leader sequence is operably linked to the 5′ terminus of the nucleic acid sequence encoding the FoPip4H. Any suitable leader sequence that is functional in the host cell of choice find use in the present invention. Exemplary leaders for filamentous fungal host cells are obtained from the genes for Aspergillus oryzae TAKA amylase, and Aspergillus nidulans triose phosphate isomerase. Suitable leaders foryeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

In some embodiments, the control sequence is also a polyadenylation sequence (i.e., a sequence operably linked to the 3′ terminus of the nucleic acid sequence and which, when transcribed, is recognized by the host cell as a signal to add polyadenosine residues to transcribed mRNA). Any suitable polyadenylation sequence that is functional in the host cell of choice may be used in the present invention. Exemplary polyadenylation sequences for filamentous fungal host cells include, but are not limited to, the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are known (See e.g., Guo and Sherman, Mol. Cell. Biol., 15:5983-5990 [1995]).

In some embodiments, the control sequence is also a signal peptide (i.e., a coding region that codes for an amino acid sequence linked to the amino terminus of a polypeptide and directs the encoded polypeptide into the cell's secretory pathway). In some embodiments, the 5′ end of the coding sequence of the nucleic acid sequence inherently contains a signal peptide coding region naturally linked in translation reading frame with the segment of the coding region that encodes the secreted polypeptide. Alternatively, in some embodiments, the 5′ end of the coding sequence contains a signal peptide coding region that is foreign to the coding sequence. Any suitable signal peptide coding region that directs the expressed polypeptide into the secretory pathway of a host cell of choice finds use for expression of the engineered polypeptide(s). Effective signal peptide coding regions for bacterial host cells are the signal peptide coding regions include, but are not limited to, those obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (See e.g., Simonen and Palva, Microbiol. Rev., 57:109-137 [1993]). In some embodiments, effective signal peptide coding regions for filamentous fungal host cells include, but are not limited to, the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to, those from the genes for Saccharomyces cerevisiae alpha-factor and Saccharomyces cerevisiae invertase.

In some embodiments, regulatory sequences are also utilized. These sequences facilitate the regulation of the expression of the polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, but are not limited to, the lac, tac, and trp operator systems. In yeast host cells, suitable regulatory systems include, but are not limited to, the ADH2 system or GAL1 system. In filamentous fungi, suitable regulatory sequences include, but are not limited to, the TAKA alpha-amylase promoter, Aspergillus niger glucoamylase promoter, and Aspergillus oryzae glucoamylase promoter.

In another aspect, the present invention is directed to a recombinant expression vector comprising a polynucleotide encoding FoPip4H polypeptide, and one or more expression regulating regions such as a promoter and a terminator, a replication origin, etc., depending on the type of hosts into which they are to be introduced. In some embodiments, the various nucleic acid and control sequences described herein are joined together to produce recombinant expression vectors that include one or more convenient restriction sites to allow for insertion or substitution of the nucleic acid sequence encoding the enzyme polypeptide at such sites. Alternatively, in some embodiments, the nucleic acid sequence of the present invention is expressed by inserting the nucleic acid sequence or a nucleic acid construct comprising the sequence into an appropriate vector for expression. In some embodiments involving the creation of the expression vector, the coding sequence is located in the vector so that the coding sequence is operably linked with the appropriate control sequences for expression.

The recombinant expression vector may be any suitable vector (e.g., a plasmid or virus), that can be conveniently subjected to recombinant DNA procedures and bring about the expression of the enzyme polynucleotide sequence. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector is one in which, when introduced into the host cell, it is integrated into the genome and replicated together with the chromosome(s) into which it has been integrated. Furthermore, in some embodiments, a single vector or plasmid, or two or more vectors or plasmids which together contain the total DNA to be introduced into the genome of the host cell, and/or a transposon is utilized.

In some embodiments, the expression vector contains one or more selectable markers, which permit easy selection of transformed cells. A “selectable marker” is a gene, the product of which provides for biocide or viral resistance, resistance to heavy metals, prototrophy to auxotrophs, and the like. Examples of bacterial selectable markers include, but are not limited to, the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to, ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof.

In another aspect, the present invention provides a host cell comprising at least one polynucleotide encoding at least one FoPip4H of the present disclosure, the polynucleotide(s) being operatively linked to one or more control sequences for expression of the at least one FoPip4H in the host cell. Host cells suitable for use in expressing the polypeptides encoded by the expression vectors of the present invention are well known in the art and include but are not limited to, bacterial cells, such as E. coli, Vibrio fluvialis, Streptomyces and Salmonella typhimurium cells; fungal cells, such as yeast cells (e.g., Saccharomyces cerevisiae or Pichia pastoris (ATCC Accession No. 201178)). Exemplary host cells also include various Escherichia coli strains (e.g., W3110 (AfhuA) and BL21). Examples of bacterial selectable markers include, but are not limited to, the dal genes from Bacillus subtilis or Bacillus licheniformis, or markers, which confer antibiotic resistance such as ampicillin, kanamycin, chloramphenicol, and or tetracycline resistance.

In some embodiments, the expression vectors of the present invention contain an element(s) that permits integration of the vector into the host cell's genome or autonomous replication of the vector in the cell independent of the genome. In some embodiments involving integration into the host cell genome, the vectors rely on the nucleic acid sequence encoding the polypeptide or any other element of the vector for integration of the vector into the genome by homologous or nonhomologous recombination.

In some alternative embodiments, the expression vectors contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements preferably contain a sufficient number of nucleotides, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are P15A ori or the origins of replication of plasmids pBR322, pUC19, pACYCI77 (which contains the P15A ori), or pACYC184 (which contains the P15A ori) permitting replication in E. coli, and pUB110, pE194, or pTA1060 permitting replication in Bacillus. Examples of origins of replication for use in a yeast host cell are the 2 micron origin of replication, ARS1, ARS4, the combination of ARSI and CEN3, and the combination of ARS4 and CEN6. The origin of replication may be one having a mutation which makes its functioning temperature-sensitive in the host cell (See e.g., Ehrlich, Proc. Natl. Acad. Sci. USA 75:1433) [1978].

In some embodiments, more than one copy of a nucleic acid sequence of the present invention is inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

Many of the expression vectors for use in the present invention are commercially available. Suitable commercial expression vectors include, but are not limited to, Novagen's® pET E. coli T7 expression vectors (Millipore Sigma) and the p3xFLAGTM™ expression vectors (Sigma-Aldrich Chemicals). Other suitable expression vectors include, but are not limited to, pBluescriptII SK(−) and pBK-CMV (Stratagene), and plasmids derived from pBR322 (Gibco BRL), pUC (Gibco BRL), pREP4, pCEP4 (Invitrogen) or pPoly (See e.g., Lathe et al., Gene 57:193-201 [1987]).

Thus, in some embodiments, a vector comprising a sequence encoding at least one variant FoPip4H is transformed into a host cell in order to allow propagation of the vector and expression of the variant FoPip4H(s). In some embodiments, the transformed host cell described above is cultured in a suitable nutrient medium under conditions permitting the expression of the variant FoPip4H(s). Any suitable medium useful for culturing the host cells finds use in the present invention, including, but not limited to minimal or complex media containing appropriate supplements. In some embodiments, host cells are grown in HTP media. Suitable media are available from various commercial suppliers or may be prepared according to published recipes (e.g., in catalogues of the American Type Culture Collection).

### Host Cells for Expression of FoPip4H Enzymes

In another aspect, the present disclosure provides a host cell comprising a polynucleotide encoding an improved FoPip4H polypeptide of the present disclosure, the polynucleotide being operatively linked to one or more control sequences for expression of the FoPip4H enzyme in the host cell. Host cells for use in expressing the FoPip4H polypeptides encoded by the expression vectors of the present invention are well known in the art and include but are not limited to, bacterial cells, such as E. coli, B. subtilis, B. licheniformis, B. megaterium, B. stearothermophilus, B. amyloliquefaciens, Lactobacillus kejir, Lactobacillus brevis, Lactobacillus minor, Streptomyces and Salmonella typhimurium cells; fungal cells, such as yeast cells (e.g., Saccharomyces cerevisiae or Pichia pastoris (ATCC Accession No. 201178)). Appropriate culture mediums and growth conditions for the above-described host cells are well known in the art.

Polynucleotides for expression of the FoPip4H polpeptides may be introduced into cells by various methods known in the art. Techniques include among others, electroporation, biolistic particle bombardment, liposome mediated transfection, calcium chloride transfection, and protoplast fusion. Various methods for introducing polynucleotides into cells will be apparent to the skilled artisan.

In some embodiments of the present invention, the filamentous fungal host cells are of any suitable genus and species, including, but not limited to Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora, Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Trametes, Tolypocladium, Trichoderma, Verticillium, and/or Volvariella, and/or teleomorphs, or anamorphs, and synonyms, basionyms, or taxonomic equivalents thereof.

In some embodiments of the present invention, the host cell is a yeast cell, including but not limited to cells of Candida, Hansenula, Saccharomyces, Schizosaccharomyces, Pichia, Kluyveromyces, or Yarrowia species. In some embodiments of the present invention, the yeast cell is Hansenula polymorpha, Saccharomyces cerevisiae, Saccharomyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some other embodiments, the host cell is a prokaryotic cell. Suitable prokaryotic cells include, but are not limited to, Gram-positive, Gram-negative and Gram-variable bacterial cells. Any suitable bacterial organism finds use in the present invention, including but not limited to Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia and Zymomonas. In some embodiments, the host cell is a species of Agrobacterium, Acinetobacter, Azobacter, Bacillus, Bifidobacterium, Buchnera, Geobacillus, Campylobacter, Clostridium, Coryne bacterium, Escherichia, Enterococcus, Erwinia, Flavobacterium, Lactobacillus, Lactococcus, Pantoea, Pseudomonas, Staphylococcus, Salmonella, Streptococcus, Streptomyces, or Zymomonas. In some embodiments, the bacterial host strain is non-pathogenic to humans. In some embodiments the bacterial host strain is an industrial strain. Numerous bacterial industrial strains are known and suitable in the present invention. In some embodiments of the present invention, the bacterial host cell is an Agrobacterium species (e.g., A. radiobacter, A. rhizogenes, and A. rubi). In some embodiments of the present invention, the bacterial host cell is an Arthrobacter species (e.g., A. aurescens, A. citreus, A. globiformis, A. hydrocarboglutamicus, A. mysorens, A. nicotianae, A. paraffineus, A. protophonniae, A. roseoparaffinus, A. sulfureus, and A. ureafaciens). In some embodiments of the present invention, the bacterial host cell is a Bacillus species (e.g., B. thuringensis, B. anthracis, B. megaterium, B. subtilis, B. lentus, B. circulans, B. pumilus, B. lautus, B. coagulans, B. brevis, B. firmus, B. alkaophius, B. licheniformis, B. clausii, B. stearothermophilus, B. halodurans, and B. amyloliquefaciens). In some embodiments, the host cell is an industrial Bacillus strain including but not limited to B. subtilis, B. pumilus, B. licheniformis, B. megaterium, B. clausii, B. stearothermophilus, or B. amyloliquefaciens. In some embodiments, the Bacillus host cells are B. subtilis, B. licheniformis, B. megaterium, B. stearothermophilus, and/or B. amyloliquefaciens. In some embodiments, the bacterial host cell is a Clostridium species (e.g., C. acetobutylicum, C. tetani E88, C. lituseburense, C. saccharobutylicum, C. perfringens, and C. beijerinckii). In some embodiments, the bacterial host cell is a Corynebacterium species (e.g., C. glutamicum and C. acetoacidophilum). In some embodiments the bacterial host cell is an Escherichia species (e.g., E. coli). In some embodiments, the host cell is Escherichia coli W3110. In some embodiments the host is Escherichia coli BL21 or BL21 (DE3). In some embodiments, the bacterial host cell is an Erwinia species (e.g., E. uredovora, E. carotovora, E. ananas, E. herbicola, E. punctata, and E. terreus). In some embodiments, the bacterial host cell is a Pantoea species (e.g., P. citrea, and P. agglomerans). In some embodiments the bacterial host cell is a Pseudomonas species (e.g., P. putida, P. aeruginosa, P. mevalonii, and P. sp. D-01 10). In some embodiments, the bacterial host cell is a Streptococcus species (e.g., S. equisimiles, S. pyogenes, and S. uberis). In some embodiments, the bacterial host cell is a Streptomyces species (e.g., S. ambofaciens, S. achromogenes, S. avermitilis, S. coelicolor, S. aureofaciens, S. aureus, S. fungicidicus, S. griseus, and S. lividans). In some embodiments, the bacterial host cell is a Zymomonas species (e.g., Z. mobilis, and Z. lipolytica).

Many prokaryotic and eukaryotic strains that find use in the present invention are readily available to the public from a number of culture collections such as American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

In some embodiments, host cells are genetically modified to have characteristics that improve protein secretion, protein stability and/or other properties desirable for expression and/or secretion of a protein. Genetic modification can be achieved by genetic engineering techniques and/or classical microbiological techniques (e.g., chemical or UV mutagenesis and subsequent selection). Indeed, in some embodiments, combinations of recombinant modification and classical selection techniques are used to produce the host cells. Using recombinant technology, nucleic acid molecules can be introduced, deleted, inhibited or modified, in a manner that results in increased yields of FoPip4H variant(s) within the host cell and/or in the culture medium. In one genetic engineering approach, homologous recombination is used to induce targeted gene modifications by specifically targeting a gene in vivo to suppress expression of the encoded protein. In alternative approaches, siRNA, antisense and/or ribozyme technology find use in inhibiting gene expression. A variety of methods are known in the art for reducing expression of protein in cells, including, but not limited to deletion of all or part of the gene encoding the protein and site-specific mutagenesis to disrupt expression or activity of the gene product. (See e.g., Chaveroche et al., Nucl. Acids Res., 28:22 e97 [2000]; Cho et al., Molec. Plant Microbe Interact., 19:7-15 [2006]; Maruyama and Kitamoto, Biotechnol. Lett., 30:1811-1817 [2008]; Takahashi et al., Mol. Gen. Genom., 272:344-352 [2004]; and You et al., Arch. Microbiol., 191:615-622 [2009], all of which are incorporated by reference herein). Random mutagenesis, followed by screening for desired mutations also finds use (See e.g., Combier et al., FEMS Microbiol. Lett., 220:141-8 [2003]; and Firon et al., Eukary. Cell 2:247-55 [2003], both of which are incorporated by reference).

Introduction of a vector or DNA construct into a host cell can be accomplished using any suitable method known in the art, including but not limited to calcium phosphate transfection, DEAE-dextran mediated transfection, PEG-mediated transformation, electroporation, or other common techniques known in the art.

In some embodiments, the engineered host cells (i.e., “recombinant host cells”) of the present invention are cultured in conventional nutrient media modified as appropriate for activating promoters, selecting transformants, or amplifying the FoPip4H polynucleotide. Culture conditions, such as temperature, pH and the like, are those previously used with the host cell selected for expression, and are well-known to those skilled in the art. As noted, many standard references and texts are available for the culture and production of many cells, including cells of bacterial, plant, animal (especially mammalian) and archebacterial origin.

In some embodiments, cells expressing the FoPip4H enzymes of the present disclosure are grown under batch or continuous fermentations conditions. Classical “batch fermentation” is a closed system, wherein the compositions of the medium are set at the beginning of the fermentation and is not subject to artificial alternations during the fermentation. A variation of the batch system is a “fed-batch fermentation” that also finds use in the present invention. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. “Continuous fermentation” is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

More than one copy of a nucleic acid sequence of the present invention may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

In some embodiments of the present invention, cell-free transcription and translation systems find use in producing the FoPip4H polypeptides. Several systems are commercially available, and the methods are well-known to those skilled in the art.

### Methods of Evolving FoPip4H Enzymes

In some embodiments, to make the FoPip4H polypeptides of the present disclosure, the FoPip4H enzyme that catalyzes the reduction reaction is obtained (or derived) from E. coli. In some embodiments, the parent polynucleotide sequence is codon optimized to enhance expression of the FoPip4H polypeptide in a specified host cell. The parental polynucleotide sequence, designated as SEQ ID NO: 3, was codon optimized for expression in E. coli and the codon-optimized polynucleotide cloned into an expression vector, placing the expression of the FoPip4H gene under the control of the T7 promoter. The T7 polymerase needed to express the gene of interest is under control of the lac promoter, and both the gene of interest and the T7 polymerase are subject to lacl repression. The presence of IPTG activates the T7 polymerase expression and eliminates the repression, resulting in production of the FoPip4H gene. Clones expressing the active FoPip4H in E. coli were identified and the genes sequenced to confirm their identity.

The FoPip4H polypeptides of the present disclosure may be obtained by subjecting the polynucleotide encoding the parent sequence to mutagenesis and/or directed evolution methods. An exemplary directed evolution technique is mutagenesis and/or DNA shuffling as described in Stemmer, 1994, Proc. Natl. Acad. Sci. USA 91:10747-10751; WO 95/22625; WO 97/20078; WO 97/35966; WO 98/27230; WO 00/42651; WO 01/75767 and U.S. Pat. No. 6,537,746. Other directed evolution procedures that can be used include, among others, staggered extension process (StEP), in vitro recombination (Zhao et al., 1998, Nat. Biotechnol. 16:258-261), mutagenic PCR (Caldwell et al., 1994, PCR Methods Appl. 3: S136-S140), and cassette mutagenesis (Black et al., 1996, Proc. Natl. Acad. Sci. USA 93:3525-3529).

The clones obtained following mutagenesis treatment are screened for FoPip4H polpeptides having a desired improved enzyme property. Measuring enzyme activity from the expression libraries can be performed using standard chemistry analytical techniques for measuring substrates and products such as UPLC-MS. Where the improved enzyme property desired is thermal stability, enzyme activity may be measured after subjecting the enzyme preparations to a defined temperature and measuring the amount of enzyme activity remaining after heat treatments. Clones containing a polynucleotide encoding a FoPip4H polypeptide are then isolated, sequenced to identify the nucleotide sequence changes (if any), and used to express the enzyme in a host cell.

Where the sequence of the polypeptide is known, the polynucleotides encoding the enzyme can be prepared by standard solid-phase methods, according to known synthetic methods. In some embodiments, fragments of up to about 100 bases can be individually synthesized, then joined (e.g., by enzymatic or chemical litigation methods, or polymerase mediated methods) to form any desired continuous sequence. For example, polynucleotides and oligonucleotides of the invention can be prepared by chemical synthesis using, e.g., the classical phosphoramidite method described by Beaucage et al., 1981, Tet. Lett. 22:1859-69, or the method described by Matthes et al., 1984, EMBO J. 3:801-05, e.g., as it is typically practiced in automated synthetic methods. According to the phosphoramidite method, oligonucleotides are synthesized, e.g., in an automatic DNA synthesizer, purified, annealed, ligated and cloned in appropriate vectors. In addition, essentially any nucleic acid can be obtained from any of a variety of commercial sources, such as The Midland Certified Reagent Company, Midland, Tex., The Great American Gene Company, Ramona, Calif., ExpressGen Inc. Chicago, Ill., Operon Technologies Inc., Alameda, Calif., and many others.

FoPip4H enzymes expressed in a host cell can be recovered from the cells and or the culture medium using any one or more of the well-known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available under the trade name CelLytic B® from Sigma-Aldrich of St. Louis Mo.

Chromatographic techniques for isolation of the FoPip4H polypeptide include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some embodiments, affinity techniques may be used to isolate the improved FoPip4H enzymes. For affinity chromatography purification, the protein sequence can be tagged with a recognition sequence to enable purification. Common tags include celluose-binding domains, poly His-tags, di-His chelates, FLAG-tags and many others that will be apparent to those having skill in the art. Antibodies can also be used as affinity purification reagents. Any antibody that specifically binds the FoPip4H polypeptide may be used.

### Processes of Using the FoPip4H Enzymes

The FoPip4H enzymes described herein can catalyze the hydroxylation of the substituted indanone substrate (II)

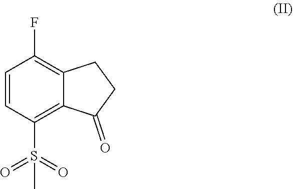

to yield the substituted hydroxyindanone (I)

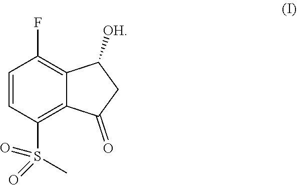 [SMILES: CS(=O)(=O)c1ccc(F)c2c1C(=O)C[C@H]2O]

Hydroxyindanone (I) is an intermediate for the synthesis of belzutifan (WELIREG). Thus, in a process for preparing belzutifan, the process can comprise a step in which substituted indanone (II) is converted to substituted hydroxyindanone (I) using a FoPip4H enzyme disclosed herein.

In the embodiments herein and illustrated in the Examples, various ranges of suitable reaction conditions that can be used in the processes, include but are not limited to, substrate loading, co-substrate loading, reductant, divalent transition metal, pH, temperature, buffer, solvent system, polypeptide loading, and reaction time. Further suitable reaction conditions for carrying out the process for biocatalytic conversion of substrate compounds to product compounds using an engineered FoPip4H polypeptide described herein can be readily optimized in view of the guidance provided herein by routine experimentation that includes, but is not limited to, contacting the engineered FoPip4H polypeptide and substrate compound under experimental reaction conditions of concentration, pH, temperature, and solvent conditions, and detecting the product compound.

Suitable reaction conditions using the engineered FoPip4H polypeptides typically comprise a co-substrate, which is used stoichiometrically in the hydroxylation reaction. Generally, the co-substrate for FoPip4H enzymes is α-ketoglutarate, also referred to as α-ketoglutaric acid and 2-oxoglutaric acid. Other analogs of α-ketoglutarate that are capable of serving as co-substrates for FoPip4 enzymes can be used. An exemplary analog that may serve as a co-substrate is 2-oxoadipate. Because the co-substrate is used stoichiometrically, the co-substrate is present at an equimolar or higher amount than that of the substrate compound, i.e., the molar concentration of co-substrate is equivalent to or higher than the molar concentration of substrate compound. In some embodiments, the suitable reaction conditions can comprise a co-substrate molar concentration of at least 1 fold, 1.5 fold, 2 fold, 3 fold 4 fold or 5 fold or more than the molar concentration of the substrate compound.

Substrate compound in the reaction mixtures can be varied, taking into consideration, for example, the desired amount of product compound, the effect of substrate concentration on enzyme activity, stability of enzyme under reaction conditions, and the percent conversion of substrate to product. In some embodiments, the suitable reaction conditions comprise a substrate compound loading of 0.5 to 200 g/L, 1 to 200 g/L, 5 to 100 g/L, or 10 to 50 g/L. The values for substrate loadings provided herein are based on the molecular weight of substituted indanone (II), however it also contemplated that the equivalent molar amounts of various hydrates and salts of substituted indanone (II) also can be used in the process.

In carrying out the FoPip4H enzyme mediated processes described herein, the engineered polypeptide may be added to the reaction mixture in the form of a purified enzyme, partially purified enzyme, whole cells transformed with gene(s) encoding the enzyme, as cell extracts and/or lysates of such cells, and/or as an enzyme immobilized on a solid support. Whole cells transformed with gene(s) encoding the engineered FoPip4H enzyme or cell extracts, lysates thereof, and isolated enzymes may be employed in a variety of different forms, including solid (e.g., lyophilized, spray-dried, and the like) or semisolid (e.g., a crude paste). The cell extracts or cell lysates may be partially purified by precipitation (ammonium sulfate, polyethyleneimine, heat treatment or the like, followed by a desalting procedure prior to lyophilization (e.g., ultrafiltration, dialysis, and the like). Any of the enzyme preparations (including whole cell preparations) may be stabilized by crosslinking using known crosslinking agents, such as, for example, glutaraldehyde or immobilization to a solid phase (e.g., Eupergit C, and the like).

The improved activity and/or stereoselectivity of the engineered FoPip4H polypeptides disclosed herein provides for processes wherein higher percentage conversion can be achieved with lower concentrations of the engineered polypeptide. In some embodiments of the process, the suitable reaction conditions comprise an engineered polypeptide amount of 1% (w/w) to 100% (w/w), from 1% (w/w) to 30% (w/w), from 2.5% (w/w) to 20% (w/w/) or from 2.5% (w/w) to 10% (w/w). In certain embodiments of the process, the suitable reaction conditions comprise an engineered polypeptide amount of 1% (w/w), 2% (w/w), 5% (w/w), 7.5% (w/w), 10% (w/w), 20% (w/w), 30% (w/w), 40% (w/w), 50% (w/w), 75% (w/w), 100% (w/w) or more of substrate compound loading.

In some embodiments, the engineered polypeptide is present at 0.01 g/L to 50 g/L; 0.1 g/L to 40 g/L; 1 g/L to 40 g/L; or 2 g/L to 10 g/L

In some embodiments, the reactions conditions also comprise a divalent transition metal capable of serving as a cofactor in the oxidation reaction. Generally, the divalent transition metal co-factor is ferrous ion, i.e., Fe⁺². The ferrous ion may be provided in various forms, such as ferrous sulfate (FeSO₄), ferrous chloride (FeCl₂), ferrous carbonate (FeCO₃), and the salts of organic acids such as citrates, lactates and fumarates. An exemplary source of ferrous sulfate is Mohr's salt, which is ferrous ammonium sulfate (NH₄)₂Fe(SO₄)₂ and is available in anhydrous and hydrated (i.e., hexahydrate) forms. While ferrous ion is the transition metal co-factor often found in the naturally occurring FoPip4 enzyme and functions efficiently in the engineered enzymes, it is to be understood that other divalent transition metals capable of acting as a co-factor can be used in the processes. In some embodiments, the divalent transition metal co-factor can comprise Mn⁺² and Cr⁺². In some embodiments, the reaction conditions can comprise a divalent transition metal cofactor, particularly Fe⁺², at a concentration of 0.1 mM to 100 mM, 0.5 mM to 80 mM, 10 mM to 60 mM or 40-60 mM.

In some embodiments, the reaction conditions can further comprise a reductant capable of reducing ferricion, Fe⁺³ to ferrous ion, Fe⁺². In some embodiments, the reductant is L-cysteine, ascorbic acid, dithiothreitol, D-cysteine, L-homocysteine, or D-cysteine ethyl ester. In some embodiments, the reductant comprises cysteine, typically L-cysteine. While cysteine is not required for the hydroxylation reaction, enzymatic activity is enhanced in its presence. Without being bound by theory, the cysteine is believed to maintain or regenerate the enzyme-Fe⁺² form, which is the active form mediating the hydroxylation reaction. Generally, the reaction conditions can comprise an cysteine concentration that corresponds proportionately to the substrate loading. In some embodiments, the cysteine is present in at least 0.1 fold, 0.2 fold 0.3 fold, or at least 0.5 fold, the molar amount of substrate. In some embodiments, the reductant, particularly L-cysteine is at a concentration of 1 mM to 100 mM, 5 mM to 80 mM or 50 to 70 mM.

In some embodiments, the reaction conditions comprise molecular oxygen, i.e., O₂. Without being bound by theory, one atom of oxygen from molecular oxygen is incorporated into the substrate compound to form the hydroxylated product compound. The O₂ may be present naturally in the reaction solution, or introduced and/or supplemented into the reaction artificially. In some embodiments, the reaction conditions can comprise forced aeration (e.g., sparging) with air, O₂ gas, or other O₂-containing gases. In some embodiments, the O₂ in the reaction can be increased by increasing the pressure of the reaction with O₂ or an O₂-containing gas. This can be done by carrying out the reaction in a vessel that can be pressurized with O₂ gas.

During the course of the reaction, the pH of the reaction mixture may change. The pH of the reaction mixture may be maintained at a desired pH or within a desired pH range. This may be done by the addition of an acid or a base, before and/or during the course of the reaction. Alternatively, the pH may be controlled by using a buffer. Accordingly, in some embodiments, the reaction condition comprises a buffer. Suitable buffers to maintain desired pH ranges are known in the art and include, by way of example and not limitation, borate, phosphate, 2-(N-morpholino) ethanesulfonic acid (MES), 3-(N-morpholino) propanesulfonic acid (MOPS), acetate, triethanolamine, and 2-amino-2-hydroxymethyl-propane-1,3-diol (Tris), and the like. In some embodiments, the buffer is phosphate. In some embodiments of the process, the suitable reaction conditions comprise a buffer (e.g., phosphate) concentration of from about 0.001 to about 0.2 M, 0.003 to about 0.1 M, or 0.005 to about 0.05 M. In some embodiments, the reaction condition comprises a buffer (e.g., phosphate) concentration of 0.001, 0.002, 0.003, 0.004, 0.005, or 0.008 M. In some embodiments, the reaction conditions comprise water as a suitable solvent with no buffer present.

In the embodiments of the process, the reaction conditions can comprise a suitable pH. The desired pH or desired pH range can be maintained by use of an acid or base, an appropriate buffer, or a combination of buffering and acid or base addition. The pH of the reaction mixture can be controlled before and/or during the course of the reaction. In some embodiments, the suitable reaction conditions comprise a solution pH from about 4 to about 10, pH from about 5 to about 10, pH from about 5 to about 9, pH from about 6 to about 9, pH from about 6 to about 8. In some embodiments, the reaction conditions comprise a solution pH of about 4, 4.5, 5, 5.5, 6, 6.5, 7, 7.5, 8, 8.5, 9, 9.5, or 10.

In the embodiments of the processes herein, a suitable temperature can be used for the reaction conditions, for example, taking into consideration the increase in reaction rate at higher temperatures, and the activity of the enzyme during the reaction time period. Accordingly, in some embodiments, the suitable reaction conditions comprise a temperature of 10° C. to 30° C., e.g., 25° C. to 30° C. In some embodiments, the temperature during the enzymatic reaction can be maintained at a specific temperature throughout the course of the reaction. In some embodiments, the temperature during the enzymatic reaction can be adjusted over a temperature profile during the course of the reaction.

The processes of the disclosure are generally carried out in a solvent. Suitable solvents include water, aqueous buffer solutions, organic solvents, polymeric solvents, and/or co-solvent systems, which generally comprise aqueous solvents, organic solvents and/or polymeric solvents. The aqueous solvent (water or aqueous co-solvent system) may be pH-buffered or unbuffered. In some embodiments, the processes using the engineered FoPip4H polypeptides can be carried out in an aqueous co-solvent system comprising an organic solvent (e.g., ethanol, isopropanol (i-PrOH), dimethyl sulfoxide (DMSO), dimethylformamide (DMF) ethyl acetate, butyl acetate, 1-octanol, heptane, octane, methyl t butyl ether (MTBE), toluene, and the like), ionic or polar solvents (e.g., 1-ethyl 4 methylimidazolium tetrafluoroborate, 1-butyl-3-methylimidazolium tetrafluoroborate, 1-butyl 3 methylimidazolium hexafluorophosphate, glycerol, polyethylene glycol, and the like). In some embodiments, the co-solvent can be a polar solvent, such as a polyol, dimethylsulfoxide (DMSO), or lower alcohol. The non-aqueous co-solvent component of an aqueous co-solvent system may be miscible with the aqueous component, providing a single liquid phase, or may be partly miscible or immiscible with the aqueous component, providing two liquid phases. Exemplary aqueous co-solvent systems can comprise water and one or more co-solvents selected from an organic solvent, polar solvent, and polyol solvent. In general, the co-solvent component of an aqueous co-solvent system is chosen such that it does not adversely inactivate the FoPip4H enzyme under the reaction conditions. Appropriate co-solvent systems can be readily identified by measuring the enzymatic activity of the specified engineered FoPip4H enzyme with a defined substrate of interest in the candidate solvent system, utilizing an enzyme activity assay, such as those described herein.

In one embodiment, the process using the FoPip4H polypeptide is performed in a co-solvent system of water and 1-octanol. The ratio of the water to octanol can 5:1 to 50:1, such as 10:1 to 40:1 or 20:1 to 40:1.

In some embodiments, the reaction conditions can include an antifoam agent, which aids in reducing or preventing formation of foam in the reaction solution, such as when the reaction solutions are mixed or sparged. Anti-foam agents include non-polar oils (e.g., minerals, silicones, etc.), polar oils (e.g., fatty acids, alkyl amines, alkyl amides, alkyl sulfates, etc.), and hydrophobic (e.g., treated silica, polypropylene, etc.), some of which also function as surfactants. Exemplary anti-foam agents include, Glanapon 2000 Konz, Y-30® (Dow Corning), poly-glycol copolymers, oxy/ethoxylated alcohols, and polydimethylsiloxanes. In some embodiments, the anti-foam can be present at 0.001% (v/v) to about 1% (v/v), 0.003 to 0.02% (v/v), or 0.005 to 0.01% (v/v).

In some embodiments, the reaction conditions can comprise a surfactant for stabilizing or enhancing the reaction. Surfactants can comprise non-ionic, cationic, anionic and/or amphiphilic surfactants. Exemplary surfactants, include by way of example and not limitation, nonyl phenoxypolyethoxylethanol (NP40), Triton X-100, polyoxyethylene-stearylamine, cetyltrimethylammonium bromide, sodium oleylamidosulfate, polyoxyethylene-sorbitanmonostearate, hexadecyldimethylamine, etc. Any surfactant that may stabilize or enhance the reaction may be employed. The concentration of the surfactant to be employed in the reaction may be generally from 0.1 to 50 mg/ml, particularly from 1 to 20 mg/ml.

The quantities of reactants used in the hydroxylase reaction will generally vary depending on the quantities of product desired, and concomitantly the amount of FoPip4H substrate employed. Those having ordinary skill in the art will readily understand how to vary these quantities to tailor them to the desired level of productivity and scale of production.

### Examples

### Example 1: Enzyme Preparation

E. coli cultures, each harboring a plasmid that encodes a FoPip4 enzyme that can be represented by amino acid sequence as set forth below in SEQ ID NOS. 1, 2, and 4-19, were diluted serially to dilutions of 104, 10-5, and 10-6 using Luria-Bretani Broth (culture media for cells) as a diluent. 100 μL of the dilutions were each spread on a petri dish containing LB agar, supplemented with 30 μg/mL of Kanamycin and 1% (w/v) glucose. The plates were placed in a 37° C. incubator overnight.

200 μL per well of Luria-Bertani Broth (culture media for cells) (500 mL LB+30 ug/mL Kanamycin+1% (w/v) glucose) was aliquoted into labeled 96-well shallow well plates. The shallow well plates were loaded into a plate stacker of a colony picker. Agar plates containing colonies sufficiently diluted so that the majority of colonies were isolated from one another (known as single colonies to those schooled in the art) were picked into unique wells of the shallow well plates. The colonies were allowed to grow overnight, at 200 rpm, at 30° C., and 85% RH.

390 μL of Terrific Broth (TB) growth media (commercially available from ThermoFisher Scientific as Catalog #A1374301) (TB+50 μg/mL Kanamycin) was aliquoted into labeled 96-well deep well subculture plates. 13 μL of the overnight growth culture was transferred from each well of the master shallow well plate into the corresponding labeled deep well subculture plates. The plates were sealed with breathable film, and the plates were shaken for 2-2.5 h at 250 rpm, at 30° C. and 85% RH. After shaking, optical density (OD₆₀₀, optical density at 600 nm wavelength) of at least one plate was measured for growth. When the OD₆₀₀ of this plate was in the range of 0.4-0.8, the deep well plates were inducted with 40 μL per well of induction media (2.2 mMIPTG, for final concentration of 0.2 mM). The plates were resealed and incubated with shaking for 18-20 h at 250 rpm, at 30° C., and 85% RH.

Alternatively, instead of TB growth media with induction media, Studier induction ZYM-5052 media was also used for expression. 390 μL of ZYM-5052 growth media (commercially available from Teknova as Catalog #3 S2000) was aliquoted into labeled 96-well deep well subculture plates. 13 μL of the overnight growth culture was transferred from each well of the master shallow well plate into the corresponding labeled deep well subculture plates. The plates were sealed with breathable film, and the plates were shaken for 20-22 h at 250 rpm, at 30° C. and 85% RH.

After incubation, all deep well plates were centrifuged for 15 min. at 4000 rpm at 4° C. Following centrifuge, the supernatant was discarded. The plates containing cell-pellets were heat-sealed and stored at −80° C.

The plates containing cell-pellets were removed from −80° C. storage and thawed at RT. A lysis buffer of 10 mM potassium phosphate pH 7.0, 0.5 mg/mL lysozyme, 0.25 mg/mL polymyxin B sulfate (PMBS), 1 units/mL DNase I and 4 mM MgSO4 was prepared. 400 μL of the lysis buffer was aliquoted into each well. The lysis mixture was shaken for 1.5-2 h at 1000 rpm on a plate shaker at RT. The lysis mixture was then centrifuged for 10 min at 4000×g at 4° C. to make the enzyme-containing lysate solution (which is in the supernatant).

### Example 2: Hydroxylase Reaction in Well Plates

5 mg of milled substrate was dispensed into each well of 1 mL round bottom well plates using a Chemspeed automated solid dispenser. A reaction buffer was prepared by mixing 448 mM alpha-ketoglutarate, 63 mM L-cysteine, 49 mM Mohr's salt in 10 mM potassium phosphate, and adjusted to a final pH of 6.5.

115 μL of the reaction buffer was added to 1 mL round bottom well plates. 10 μL of the enzyme-containing lysate solution of Example 1 was then added. The plate was sealed with a breathable seal and shaken overnight at 30° C., 250 rpm and 85% RH.

For thermostability tests, the same reaction set-up as above is used but 50 μL of the enzyme-containing lysate solutions of Example 1 were first transferred to Biorad hardshell PCR plates and heat-treated for 10 minutes at the T50 of the enzyme before transferring 10 μL of heat-teated lysate into the reaction plate to initiate reaction. Heat-treated supernatants were assayed for residual activity compared to non-treated supernatants.

After overnight shaking, 250 μL of DMSO was added to each well of the reaction plates. The plates were heat sealed and shook for 10 minutes at 1000 rpm, RT. 180 μL of a mixture of 50% ACN/water was aliquoted into filter stacks (filter plates on top of the round bottom plates, with 0.20 μM hydrophilic PTFE, commercially available from Millipore MSRLN2250). The reaction plates were unsealed. 10 μL of reaction mixture from the reaction plates was transferred into the corresponding filter plates with a receiving plate underneath. The plates were then centrifuged for 3 min at 4000 rpm at 4° C. The filter plates were removed, and the clear solutions in the receiving plates were heat sealed.

The filtered solutions were analyzed by ultra-performance liquid chromatography (UPLC), using a high throughput screening method to monitor the substrate, the product, and product overoxidation peak areas. The structure of the overoxidized product is shown below.

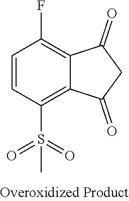 [SMILES: CS(=O)(=O)c1ccc(F)c2c1C(=O)CC2=O]

UPLC was conducted on an Acquity UPLC@HSS T3 1.8 μm 2.1×50 mm column using the following gradient method where mobile phase component A is water with 0.1% TFA and component B is ACN with 0.1% TFA: 12% B from 0-0.7 min, gradient from 12% to 95% B from 0.7-0.8 min, hold at 95% B from 0.8-1.2 min, gradient from 95% to 12% B from 1.2-1.25 min, and hold at 12% B from 1.25-1.5 min. Column temperature was set to 40° C., flow rate to 0.75 mL/min, and absorbance at 254 and 290 nm were measured. The starting material eluted at 1.1 min, the desired product at 0.61 min and the undesired overoxidation product at 0.48 min.

In addition to UPLC analysis, plate reader analysis was also used as a method to measure overoxidation. After the overnight reaction, plates were centrifuged for 10 minutes at 1000×g. 190 μL of water was aliquoted into each well of Greiner 96-well black plates. 10 μL of the overnight reaction was transferred to the Greiner plates with water. Absorbance at 300 and 400 nm were measured, with the ratio of 300 to 400 nm used as a proxy for relative amounts of desired product to undesired overoxidation.

### Example 3: Enzyme Preparation in Shaker Flasks

5 mL of Luria-Bertani Broth (culture media for cells) (250 mL LB+30 μg/mL Kanamycin+1% glucose) was inoculated with 10 μL of E. coli cells, each harboring a plasmid that encodes a FoPip4H enzyme that can be represented by amino acid sequence as set forth below in SEQ ID NOS. 1, 2, and 4-19, that had been stored at −80° C. in 20% glycerol and aliquoted into labeled 15 mL cell culture tubes. The cell culture tubes were sealed and incubated with shaking for 20-24 h at 250 rpm, at 30° C.

After overnight growth, the overnight growth cultures (2-5 mL of cell culture (having a starting OD₆₀₀ of 0.2)) were added to 250 mL of Terrific Broth (TB) growth media (commercially available from ThermoFisher Scientific as Catalog #A1374301) (TB+30 μg/mL Kanamycin) to a final volume of 250 mL. The flasks were shaken for 3-4 h at 250 rpm, at 30° C. After shaking, OD₆₀₀ was measured for growth until OD₆₀₀ reached 0.4-0.6. At this point, 0.2 mM of IPTG (50 μL of 1M IPTG) was added to the culture to induce expression, and the culture was allowed to grow 20-24 h at 250 rpm, at 30° C.

After the additional growth period, the cultures were transferred to a centrifuge bottle of known weight and centrifuged for 20 min. at 4000 rpm at 4° C. Following centrifugation, the supernatant was discarded, and the remaining cell pellet in the bottle was weighed. The weight of the cell pellet was calculated by subtracting the known weight of the bottle, and the cell pellet was resuspended in 5 mL of 10 mM potassium phosphate buffer (pH=7) per gram of well-cell pellet.

The cells from the resuspended cell pellets were lysed using a microfluidizer, and the cell lysate was collected and centrifuged for 60 min. at 10000 rpm at 4° C. The clarified supernatant was transferred to a petri dish and frozen at −80° C. for approximately 2 h. Samples were lyophilized using a standard automated protocol.

### Example 4: Hydroxylase Reaction in Vials, Conversion and Selectivity Determinations

With selected variants of the FoPip4H enzyme, the hydroxylase reaction was performed on the indanone substrate of Formula (II) in vials to further evaluate the variants for their conversion and selectivity (i.e., enantioselectivity). To a 50 mL vial were added a-ketoglutaric acid (2.8 g, 19.2 mmol), Mohr's salt (0.86 g, 2.2 mmol), and cysteine (0.34 g, 2.8 mmol). The mixture was dissolved in 25 mL of water, the pH was adjusted to 6.0 with 5 N

NaOH, and the final volume was brought to 44.5 mL with water to provide the reaction solution. To a fresh 50 mL vial were added the indanone substrate of Formula (II) (0.2 g, 0.88 mmol) and FoPip4H enzyme (15 mg, 7.5 wt %, from Example 3) to provide the solids vial. 4.45 mL of the reaction solution was transferred to the solids vial and 0.3 mL 1-octanol was added. The solution was stirred for 24 hours prior to sampling.

Conversion was determined by comparing product and starting material peak areas on an Agilent UPLC equipped with a Waters Aquity HSS T3 column. The mobile phases were 0.1% phosphoric acid in water and acetonitrile. Product and starting material were identified by comparison to pure standards.

Enantioselectivity (ee) was determined by comparing peak areas of enantiomers separated on a Waters SFC column. The SFC column was equipped with a Chiralpak AD-3 column and used methanol and CO₂ as the mobile phase. Enantiomers were determined by comparison to pure standards.

### Example 5: Determination of T₅₀

15 mg of each selected FoPip4H enzyme was dissolved in 10 mM potassium phosphate buffer at pH 6.0. Separate solutions were held at temperatures ranging from 40-65° C. for one hour followed by centrifugation to clarify the mixture. Solutions were then used to set up assays as described in Example 2.

### Example 6: Preparation of (R)-4-fluoro-3-hydroxy-7-(methylsulfonyl)-2,3-dihydro-1H-inden-1-one (I)

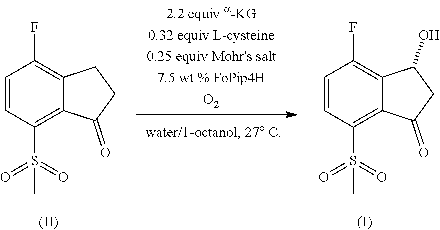

Water (34.5 L, 23 vol.) was added to a 100 L reactor and adjusted to 20-30° C. Following addition of alpha-ketoglutaric acid (2.11 kg, 14.46 mol, 2.20 equiv.), the solution was sparged with N₂ until dissolved oxygen (dO) level was <2%. L-cysteine (0.26 kg, 2.10 mol, 0.32 equiv.) was added, and pH was adjusted to 6.8 with 10N NaOH. Mohr's salt (0.64 kg, 1.64 mol, 0.25 equiv.) was added, followed by 5 N NaOH to adjust to pH 6.0. 1-octanol (1.13 L, 0.75 vol) was added, followed by lyophilized fermentation powder containing FoPip4H (0.11 kg, 7.5 wt %) and then sulfone (II) (1.5 kg, 6.57 mol, 1.00 equiv.). The reaction dO level was brought to 100%, and the suspension was stirred for 24 to 48 h, after which sulfuric acid was added to adjust to pH 4.7. To this mixture was added (NH4) 2 (SO4) (9.0 kg, 68.1 mol) and 50% MeCN in toluene (42 L, 28 vol.) followed by CELITE (3.0 kg) and the mixture heated to 45° C. for 2 h, then cooled to 25° C. The mixture was filtered and the organic layer was separated. The filtered cake was washed with 25% MeCN in toluene (15 L, 10 vol.) twice. The aqueous layer was back-extracted with the cake wash solvent twice and the organic layers combined and washed with water (1.1 L, 0.75 vol). The organic layer was concentrated to 10 vol. at 50° C. under vacuum then stirred for 2 h at 55° C., cooled to 25° C. over 4 h and stirred for an additional 10 hrs. The slurry was filtered and washed with 5% MeCN in toluene (1.5 L, 1 vol.) three times. The cake was dried under vacuum to afford 1.39 kg of hydroxysulfone (I) (99 wt %, 5.63 mol, 86% yield). 1H NMR (599.90 MHZ, DMSO-d₆) δ 8.11 (dd, J=8.4 and 4.4 Hz, 1H, CH), 7.78 (t, J=8.5 Hz, 1H, CH), 5.97 (d, J=7.3 Hz, 1H, OH), 5.46 (td, J=7.1 and 2.2 Hz, 1H, CH), 3.42 (s, 3H, CH3), 3.20 (dd, J=18.8 and 6.8 Hz, 1H, CH′H″), 2.59 (dd, J=18.8 and 2.2 Hz, 1H, CH′H″) ppm. ¹³C {¹H} NMR (150.85 MHz, DMSO-d₆) δ 200.17 (s, C═O), 162.85 (d, J_{CF}=259.9 Hz, CF), 145.05 (d, J_{CF}=19.2 Hz, C), 136.02 (d, J_{CF}=5.1 Hz, C), 133.24 (d, J_{CF}=4.0 Hz, C), 132.32 (d, J_{CF}=8.5 Hz, CH), 121.39 (d, J_{CF}=21.0 Hz, CH), 63.85 (s, CH), 47.22 (s, CH2), 42.65 (s, CH3) ppm. ¹⁹F NMR (564.47 MHz, DMSO-d₆) δ-110.86 (dd, J_{HF}=8.6 and 4.5 Hz, 1F) ppm.

SEQUENCES:

MGSHHHHHHHHGSAALNADTLDMSLFFGTPSQKQDFCDSLLRLLKKRGGVKLINHPIPS
TSIHELFAQTKRFFNLPLETKMLAKHPPQANPNRGYSFVGQENVANISGYEKGLGPLKT
RDIKETVDFGSANDELVDNLWVPEEELPGFRSFMEGFYELAFKTEMQLLEALAIALGVS
PDHLKSLHNRAENEFRILHYPAIPASELADGTATRIAEHTDFGTITMLFQDSVGGLQVED
QENLGTFNNVESASPTDIILNIGDSLQRLTNDTFKAACHRVTYPPSIKAGDGEQVIPERYSI
AYFAKPNRSASLFPLKEFIEEGVPCKYEDVTAWEWNNRRIEKLFSAEAKA (SEQ ID
NO: 1)
MGSHHHHHHHHGSAALNADTLDMSLFFGTPSQKQDFCDSLLRLLKARGVVKLINHPIPS
ESIHELFAQTKRFFNLPLETKMLAKHPEQALPARGYAFVGQENVANISGYEKGLPPLKTR
DIKETVDFGSANDEKYDNLWVPEEELPGFRSFMEGFYELAFKTEMQILEALAIALGVSPD
HLKSLHNRAHSELRILHYPAIPASELADGTATRIAEHTDFGSITMLFQDGVGGLQVEDQE
NLGTFNNVESASPTDIILNIGDSLQRLTNDTFKAACHRVTWPPSIKDGDGSEVIPERYSVA
YFVKPNRSASLFPLKEFIEEGVPPKYEDLTFEEWNNRRIEKLFSAEAKA (SEQ ID NO: 2)
ATGGGATCTCACCATCATCATCACCATCACCACGGCTCCGCGGCTCTGAATGCAGAT
ACCCTTGACATGTCATTGTTTTTCGGTACACCTTCGCAAAAACAGGATTTCTGTGATA
GCCTGTTACGTCTTCTGAAGGCGCGCGGCGTTGTGAAGTTAATCAATCATCCGATCC
CATCGGAATCTATTCATGAGTTATTCGCACAAACGAAACGCTTTTTTAACTTACCCTT
AGAAACCAAGATGCTGGCAAAGCATCCCGAGCAAGCGTTGCCCGCCCGTGGCTATG
CGTTTGTTGGACAGGAGAATGTGGCAAACATCTCTGGCTATGAGAAAGGTCTGCCGC
CCCTGAAGACCCGCGATATTAAGGAAACTGTAGATTTCGGTTCTGCAAACGACGAG
AAGTATGACAATCTTTGGGTGCCGGAAGAGGAACTTCCGGGTTTCCGCAGTTTCATG
GAAGGTTTCTACGAGCTTGCATTCAAGACTGAAATGCAGATTTTGGAAGCCCTTGCA
ATCGCACTTGGCGTAAGTCCAGACCATCTGAAAAGCTTGCACAACCGTGCGCATAG
CGAATTGCGTATCTTACACTATCCCGCCATTCCTGCTAGTGAACTTGCTGATGGCACC
GCGACGCGCATTGCCGAGCACACGGACTTTGGGAGCATTACCATGTTGTTTCAAGAC
GGGGTCGGTGGTCTGCAAGTTGAAGACCAGGAAAATTTGGGCACCTTTAACAATGTT
GAGTCGGCATCGCCTACTGACATCATCTTGAATATCGGAGACAGTTTGCAGCGCCTG
ACCAACGACACATTTAAGGCCGCATGCCACCGCGTCACTTGGCCACCGAGTATTAA
AGATGGAGATGGGAGCGAAGTAATTCCCGAACGTTACAGTGTTGCGTACTTCGTGA
AACCTAATCGTAGTGCCAGCTTATTCCCGTTAAAGGAGTTCATCGAAGAGGGTGTAC
CTCCCAAATATGAAGACCTGACGTTTGAAGAGTGGAACAATCGTCGCATCGAGAAA
CTGTTCTCTGCGGAAGCAAAAGCGTAA (SEQ ID NO: 3)
MGSHHHHHHHHGSAALNADTLDMSLFFGTPSQKQDFCDSLLRLLKKRGGVKLINHPIPS
TSIHELFAQTKRFFNLPLETKMLAKHPPQANPNRGYLFVGQENVANISGYEKGLGPLKT
RDIKETVDFGSANDELVDNLWVPEEELPGFRSFMEGFYELAFKTEMQLLEALAIALGVS
PDHLKSLHNRAENEFRILHYPAIPASELADGTATRIAEHTDFGTITMLFQDSVGGLQVED
QENLGTFNNVESASPTDIILNIGDSLQRLTNDTFKAACHRVTYPPSIKAGDGEQVIPERYSI
AYFAKPNRSASLFPLKEFIEEGVPCKYEDVTAWEWNNRRIEKLFSAEAKA (SEQ ID NO: 4)
MGSHHHHHHHHGSAALNADTLDMSLFFGTPSQKQDFCDSLLRLLKKRGGVKLINHPIPS
TSIHELFAQTKRFFNLPLETKMLAKHPPQANPARGYLFVGQENVANISGYEKGLGPLKT
RDIKETVDFGSANDELVDNLWVPEEELPGFRSFMEGFYELAFKTEMQLLEALAIALGVS
PDHLKSLHNRAENELRILHYPAIPASELADGTATRIAEHTDFGTITMLFQDSVGGLQVED
QENLGTFNNVESASPTDIILNIGDSLQRLTNDTFKAACHRVTYPPSIKAGDGEQVIPERYSI
AYFVKPNRSASLFPLKEFIEEGVPCKYEDVTAWEWNNRRIEKLFSAEAKA (SEQ ID
NO: 5)
MGSHHHHHHHHGSAALNADTLDMSLFFGTPSQKQDFCDSLLRLLKKRGGVKLINHPIPS
TSIHELFAQTKRFFNLPLETKMLAKHPPQALPARGYLFVGQENVANISGYEKGLGPLKTR
DIKETVDFGSANDELYDNLWVPEEELPGFRSFMEGFYELAFKTEMQLLEALAIALGVSP
DHLKSLHNRAENELRILHYPAIPASELADGTATRIAEHTDFGTITMLFQDSVGGLQVEDQ
ENLGTFNNVESASPTDIILNIGDSLQRLTNDTFKAACHRVTYPPSIKAGDGEQVIPERYSIA
YFVKPNRSASLFPLKEFIEEGVPCKYEDVTAWEWNNRRIEKLFSAEAKA (SEQ ID NO: 6)
MGSHHHHHHHHGSAALNADTLDMSLFFGTPSQKQDFCDSLLRLLKKRGGVKLINHPIPS
TSIHELFAQTKRFFNLPLETKMLAKHPPQALPARGYLFVGQENVANISGYEKGLGPLKTR
DIKETVDFGSANDELYDNLWVPEEELPGFRSFMEGFYELAFKTEMQLLEALAIALGVSP
DHLKSLHNRAENELRILHYPAIPASELADGTATRIAEHTDFGTITMLFQDSVGGLQVEDQ
ENLGTFNNVESASPTDIILNIGDSLQRLINDTFKAACHRVTYPPSIKAGDGEQVIPERYSIA
YFVKPNRSASLFPLKEFIEEGVPCKYEDVTAEEWNNRRIEKLFSAEAKA (SEQ ID NO: 7)
MGSHHHHHHHHGSAALNADTLDMSLFFGTPSQKQDFCDSLLRLLKARGGVKLINHPIPS
TSIHELFAQTKRFFNLPLETKMLAKHPPQALPARGYLFVGQENVANISGYEKGLSPLKTR
DIKETVDFGSANDELYDNLWVPEEELPGFRSFMEGFYELAFKTEMQLLEALAIALGVSP
DHLKSLHNRAENELRILHYPAIPASELADGTATRIAEHTDFGTITMLFQDSVGGLQVEDQ
ENLGTFNNVESASPTDIILNIGDSLQRLTNDTFKAACHRVTYPPSIKAGDGEQVIPERYSV
AYFVKPNRSASLFPLKEFIEEGVPCKYEDVTAEEWNNRRIEKLFSAEAKA (SEQ ID NO: 8)
MGSHHHHHHHHGSAALNADTLDMSLFFGTPSQKQDFCDSLLRLLKARGGVKLINHPIPS
TSIHELFAQTKRFFNLPLETKMLAKHPPQALPARGYLFVGQENVANISGYEKGLSPLKTR
DIKETVDFGSANDELYDNLWVPEEELPGFRSFMEGFYELAFKTEMQLLEALAIALGVSP
DHLKSLHNRAHNELRILHYPAIPASELADGTATRIAEHTDFGTITMLFQDSVGGLQVEDQ
ENLGTFNNVESASPTDIILNIGDSLQRLTNDTFKAACHRVTYPPSIKAGDGEQVIPERYSV
AYFVKPNRSASLFPLKEFIEEGVPCKYEDVTAEEWNNRRIEKLFSAEAKA (SEQ ID NO: 9)
MGSHHHHHHHHGSAALNADTLDMSLFFGTPSQKQDFCDSLLRLLKARGGVKLINHPIPS
TSIHELFAQTKRFFNLPLETKMLAKHPPQPLPARGYLFVGQENVANISGYEKGLSPLKTR
DIKETVDFGSANDELYDNLWVPEEELPGFRSFMEGFYELAFKTEMQLLEALAIALGVSP
DHLKSLHNRAHNELRILHYPAIPASELADGTATRIAEHTDFGSITMLFQDSVGGLQVEDQ
ENLGTFNNVESASPTDIILNIGDSLQRLTNDTFKAACHRVTYPPSIKAGDGEQVIPERYSV
AYFVKPNRSASLFPLKEFIEEGVPCKYEDVTAEEWNNRRIEKLFSAEAKA (SEQ ID
NO: 10)
MGSHHHHHHHHGSAALNADTLDMSLFFGTPSQKQDFCDSLLRLLKARGGVKLINHPIPS
TSIHELFAQTKRFFNLPLETKMLAKHPPQALPARGYSFVGQENVANISGYEKGLSPLKTR
DIKETVDFGSANDELYDNLWVPEEELPGFRSFMEGFYELAFKTEMQLLEALAIALGVSP
DHLKSLHNRAHNELRILHYPAIPASELADGTATRIAEHTDFGSITMLFQDSVGGLQVEDQ
ENLGTFNNVESASPTDIILNIGDSLQRLTNDTFKAACHRVTYPPSIKAGDGEQVIPERYSV
AYFVKPNRSASLFPLKEFIEEGVPCKYEDVTAEEWNNRRIEKLFSAEAKA (SEQ ID
NO: 11)
MGSHHHHHHHHGSAALNADTLDMSLFFGTPSQKQDFCDSLLRLLKARGGVKLINHPIPS
ESIHELFAQTKRFFNLPLETKMLAKHPPQALPARGYSFVGQENVANISGYEKGLSPLKTR
DIKETVDFGSANDELYDNLWVPEEELPGFRSFMEGFYELAFKTEMQLLEALAIALGVSP
DHLKSLHNRAHNELRILHYPAIPASELADGTATRIAEHTDFGSITMLFQDSVGGLQVEDQ
ENLGTFNNVESASPTDIILNIGDSLQRLTNDTFKAACHRVTYPPSIKAGDGEQVIPERYSV
AYFVKPNRSASLFPLKEFIEEGVPCKYEDVTAEEWNNRRIEKLFSAEAKA (SEQ ID
NO: 12)
MGSHHHHHHHHGSAALNADTLDMSLFFGTPSQKQDFCDSLLRLLKARGGVKLINHPIPS
ESIHELFAQTKRFFNLPLETKMLAKHPPQALPARGYAFVGQENVANISGYEKGLSPLKTR
DIKETVDFGSANDELYDNLWVPEEELPGFRSFMEGFYELAFKTEMQLLEALAIALGVSP
DHLKSLHNRAHNELRILHYPAIPASELADGTATRIAEHTDFGSITMLFQDSVGGLQVEDQ
ENLGTFNNVESASPTDIILNIGDSLQRLTNDTFKAACHRVTYPPSIKAGDGEQVIPERYSV
AYFVKPNRSASLFPLKEFIEEGVPCKYEDVTAEEWNNRRIEKLFSAEAKA (SEQ ID
NO: 13)
MGSHHHHHHHHGSAALNADTLDMSLFFGTPSQKQDFCDSLLRLLKARGGVKLINHPIPS
ESIHELFAQTKRFFNLPLETKMLAKHPPQALPARGYAFVGQENVANISGYEKGLPPLKTR
DIKETVDFGSANDEKYDNLWVPEEELPGFRSFMEGFYELAFKTEMQLLEALAIALGVSP
DHLKSLHNRAHNELRILHYPAIPASELADGTATRIAEHTDFGSITMLFQDGVGGLQVEDQ
ENLGTFNNVESASPTDIILNIGDSLQRLTNDTFKAACHRVTYPPSIKDGDGEQVIPERYSV
AYFVKPNRSASLFPLKEFIEEGVPCKYEDVTAEEWNNRRIEKLFSAEAKA (SEQ ID
NO: 14)
MGSHHHHHHHHGSAALNADTLDMSLFFGTPSQKQDFCDSLLRLLKARGVVKLINHPIPS
ESIHELFAQTKRFFNLPLETKMLAKHPPQALPARGYAFVGQENVANISGWEKGLPPLKT
RDIKETVDFGSANDEKYDNLWVPEEELPGFRSFMEGFYELAFKTEMQLLEALAIALGVS
PDHLKSLHNRAHNELRILHYPAIPASELADGTATRIAEHTDFGSITMLFQDGVGGLQVED
QENLGTFNNVESASPTDIILNIGDSLQRLTNDTFKAACHRVTWPPSIKDGDGTQVIPERYS
VAYFVKPNRSASLFPLKEFIEEGVPCKYEDLTAEEWNNRRIEKLFSAEAKA (SEQ ID
NO: 15)
MGSHHHHHHHHGSAALNADTLDMSLFFGTPSQKQDFCDSLLRLLKARGVVKLINHPIPS
ESIHELFAQTKRFFNLPLETKMLAKHPEQALPARGYAFVGQENVANISGYEKGLPPLKTR
DIKETVDFGSANDEKYDNLWVPEEELPGFRSFMEGFYELAFKTEMQLLEALAIALGVSP
DHLKSLHNRAHSELRILHYPAIPASELADGTATRIAEHTDFGSITMLFQDGVGGLQVEDQ
ENLGTFNNVESASPTDIILNIGDSLQRLTNDTFKAACHRVTWPPSIKDGDGSEVIPERYSV
AYFVKPNRSASLFPLKEFIEEGVPPKYEDLTAEEWNNRRIEKLFSAEAKA (SEQ ID
NO: 16)
MGSHHHHHHHHGSAALNADTLDMSLFFGTPSQKQDFCDSLLRLLKARGVVKLINHPIPS
ESIHELFAQTKRFFNLPLETKMLAKHPEQALPARGYAFVGQENVANISGYEKGLPPLKTR
DIKETVDFGSANDEKYDNLWVPEEELPGFRSFMEGFYELAFKTEMQILEALAIALGVSPD
HLKSLHNRAHSELRILHYPAIPASELADGTATRIAEHTDFGSITMLFQDGVGGLQVEDQE
NLGTFNNVESASPTDIILNIGDSLQRLTNDTFKAACHRVTWPPSIKDGDGSEVIPERYSVA
YFVKPNRSASLFPLKEFIEEGVPPKYEDLTAEEWNNRRIEKLFSAEAKA (SEQ ID NO: 17)
MGSHHHHHHHHGSAALNADTLDMSLFFGTPSQKQDFCDSLLRLLKAKGVVKLINHPIP
AESIKELFAQTKRFFNLPLETKMLAKHPEQALPARGYAFVGQENVANISGYEKGLPPLK
TRDIKETVDFGSANDEKYDNLWVPEEELPGFRSFMEGFYELAFKTEMQILEALAIALGVS
PDHLKSLHNGAHSELRILHYPAIPASELADGTATRIAEHTDFGSITMLFQDGVGGLQVED
QENLGTFNNVESASPTDIILNIGDSLQRLTNDTFKAACHRVTWPPSIKDGDGSEVIPERYS
VAYFVKPNRSASLFPLKEFIEEGVPPKYEDLTFEEWNNRRIEKLFSAEAKA (SEQ ID
NO: 18)
MGSHHHHHHHHGSAALNADTLDMSLFFGTPSQKQDFCDSLLRLLKAKGVVKLINHPIP
AESIKELFAQTKRFFNLPLETKMLAKHPEQALPARGYAFVGQENVANISGYEKGLPPLK
TRDIKETVDFGSANDEKYDNLWVPEEELPGFRSFMEGFYELAFKTEMQILEALAIALGVS
PDHLKSLHNSAHSELRILHYPAIPASELADGTATRIAEHTDFGSITMLFQDGVGGLQVED
QENLGTFNNVESASPTDIILNIGDSLQRLTNDTFRAACHRVTWPPSIKDGDGSEVIPERYS
VAYFVKPNRSASLFPLKEFIEEGVPPKYEDLTFEEWNNRRIEKLFSAEAKA (SEQ ID
NO: 19)

It will be appreciated that various of the above-discussed and other features and functions, or alternatives thereof, may be desirably combined into many other different systems or applications. Also that various presently unforeseen or unanticipated alternatives, modifications, variations, or improvements therein may be subsequently made by those skilled in the art which are also intended to be encompassed by the following claims.

1. A polypeptide comprising an amino acid sequence having at least a 90% sequence identity to SEQ ID NO: 2.

2. The polypeptide according to claim 1, wherein the amino acid sequence has at least a 95% sequence identity to SEQ ID NO: 2.

3. The polypeptide according to claim 1, wherein the amino acid sequence has at least a 98% sequence identity to SEQ ID NO: 2.

4. The polypeptide according to claim 1, wherein the amino acid sequence consists of SEQ ID NO: 2.

5. The polypeptide according to claim 1, consisting of SEQ ID NO: 2.

6. A polynucleotide encoding the polypeptide according to claim 1.

7. The polynucleotide of claim 6, wherein the polynucleotide comprises SEQ ID NO: 3.

8. An expression vector comprising the polynucleotide according to claim 6, operably linked to one or more control sequences suitable for directing expression of the encoded polypeptide in a host cell.

9. The expression vector of claim 8, wherein the control sequence comprises a promoter.

10. The expression vector of claim 9, wherein the promoter comprises an E. coli promoter.

11. A host cell comprising the expression vector of claim 8.

12. The host cell of claim 11, wherein the host cell is E. coli.

13. A process for preparing a compound according to Formula (I) 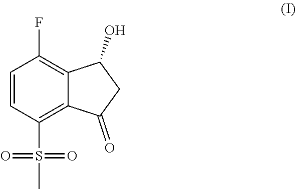 [SMILES: CS(=O)(=O)c1ccc(F)c2c1C(=O)C[C@H]2O]
in at least 60% ee, the process comprising contacting the indanone according to Formula (II) 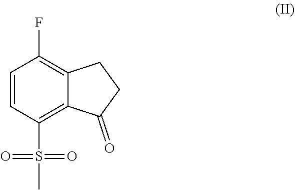
with α-ketoglutarate in the presence of the polypeptide according to claim 1 to provide the compound of Formula (I).

14. The process of claim 13, further comprising a reductant selected from the group consisting of L-cysteine, ascorbic acid, dithiothreitol, D-cysteine, L-homocysteine, and D-cysteine ethyl ester.

15. The process of claim 14, further comprising a buffer selected from the group consisting of phosphate buffer, 2-morpholinoethanesulfonic acid, Bis Tris, PIPES, citrate, bicine, and TEOA.

16. The process of claim 14, further comprising iron salt selected from the group consisting of Mohr's salt ((NH₄)₂Fe(SO₄)₂ 6H₂O), and iron chloride.

17. The process of claim 13, wherein the FoPip4 hydroxylase comprises an amino acid sequence having at least 95% sequence identity to SEQ ID NO:2.

18. The process of claim 13, wherein the compound of Formula (I) is prepared in at least 95% ee.


SEQUENCE LISTING (19 sequences)

SEQ ID NO: 1 - protein, 349 aa, synthetic construct
MGSHHHHHHHHGSAALNADTLDMSLFFGTPSQKQDFCDSLLRLLKKRGGVKLINHPIPSTSIHELFAQTKRFFNLPLETKMLAKHPPQANPNRGYSFVGQENVANISGYEKGLGPLKTRDIKETVDFGSANDELVDNLWVPEEELPGFRSFMEGFYELAFKTEMQLLEALAIALGVSPDHLKSLHNRAENEFRILHYPAIPASELADGTATRIAEHTDFGTITMLFQDSVGGLQVEDQENLGTFNNVESASPTDIILNIGDSLQRLTNDTFKAACHRVTYPPSIKAGDGEQVIPERYSIAYFAKPNRSASLFPLKEFIEEGVPCKYEDVTAWEWNNRRIEKLFSAEAKA

SEQ ID NO: 2 - protein, 349 aa, synthetic construct
MGSHHHHHHHHGSAALNADTLDMSLFFGTPSQKQDFCDSLLRLLKARGVVKLINHPIPSESIHELFAQTKRFFNLPLETKMLAKHPEQALPARGYAFVGQENVANISGYEKGLPPLKTRDIKETVDFGSANDEKYDNLWVPEEELPGFRSFMEGFYELAFKTEMQILEALAIALGVSPDHLKSLHNRAHSELRILHYPAIPASELADGTATRIAEHTDFGSITMLFQDGVGGLQVEDQENLGTFNNVESASPTDIILNIGDSLQRLTNDTFKAACHRVTWPPSIKDGDGSEVIPERYSVAYFVKPNRSASLFPLKEFIEEGVPPKYEDLTFEEWNNRRIEKLFSAEAKA

SEQ ID NO: 3 - DNA, 1050 bp, synthetic construct
  source 1..1050: mol_type = other DNA
atgggatctcaccatcatcatcaccatcaccacggctccgcggctctgaatgcagatacccttgacatgtcattgtttttcggtacaccttcgcaaaaacaggatttctgtgatagcctgttacgtcttctgaaggcgcgcggcgttgtgaagttaatcaatcatccgatcccatcggaatctattcatgagttattcgcacaaacgaaacgcttttttaacttacccttagaaaccaagatgctggcaaagcatcccgagcaagcgttgcccgcccgtggctatgcgtttgttggacaggagaatgtggcaaacatctctggctatgagaaaggtctgccgcccctgaagacccgcgatattaaggaaactgtagatttcggttctgcaaacgacgagaagtatgacaatctttgggtgccggaagaggaacttccgggtttccgcagtttcatggaaggtttctacgagcttgcattcaagactgaaatgcagattttggaagcccttgcaatcgcacttggcgtaagtccagaccatctgaaaagcttgcacaaccgtgcgcatagcgaattgcgtatcttacactatcccgccattcctgctagtgaacttgctgatggcaccgcgacgcgcattgccgagcacacggactttgggagcattaccatgttgtttcaagacggggtcggtggtctgcaagttgaagaccaggaaaatttgggcacctttaacaatgttgagtcggcatcgcctactgacatcatcttgaatatcggagacagtttgcagcgcctgaccaacgacacatttaaggccgcatgccaccgcgtcacttggccaccgagtattaaagatggagatgggagcgaagtaattcccgaacgttacagtgttgcgtacttcgtgaaacctaatcgtagtgccagcttattcccgttaaaggagttcatcgaagagggtgtacctcccaaatatgaagacctgacgtttgaagagtggaacaatcgtcgcatcgagaaactgttctctgcggaagcaaaagcgtaa

SEQ ID NO: 4 - protein, 349 aa, synthetic construct
MGSHHHHHHHHGSAALNADTLDMSLFFGTPSQKQDFCDSLLRLLKKRGGVKLINHPIPSTSIHELFAQTKRFFNLPLETKMLAKHPPQANPNRGYLFVGQENVANISGYEKGLGPLKTRDIKETVDFGSANDELVDNLWVPEEELPGFRSFMEGFYELAFKTEMQLLEALAIALGVSPDHLKSLHNRAENEFRILHYPAIPASELADGTATRIAEHTDFGTITMLFQDSVGGLQVEDQENLGTFNNVESASPTDIILNIGDSLQRLTNDTFKAACHRVTYPPSIKAGDGEQVIPERYSIAYFAKPNRSASLFPLKEFIEEGVPCKYEDVTAWEWNNRRIEKLFSAEAKA

SEQ ID NO: 5 - protein, 349 aa, synthetic construct
MGSHHHHHHHHGSAALNADTLDMSLFFGTPSQKQDFCDSLLRLLKKRGGVKLINHPIPSTSIHELFAQTKRFFNLPLETKMLAKHPPQANPARGYLFVGQENVANISGYEKGLGPLKTRDIKETVDFGSANDELVDNLWVPEEELPGFRSFMEGFYELAFKTEMQLLEALAIALGVSPDHLKSLHNRAENELRILHYPAIPASELADGTATRIAEHTDFGTITMLFQDSVGGLQVEDQENLGTFNNVESASPTDIILNIGDSLQRLTNDTFKAACHRVTYPPSIKAGDGEQVIPERYSIAYFVKPNRSASLFPLKEFIEEGVPCKYEDVTAWEWNNRRIEKLFSAEAKA

SEQ ID NO: 6 - protein, 349 aa, synthetic construct
MGSHHHHHHHHGSAALNADTLDMSLFFGTPSQKQDFCDSLLRLLKKRGGVKLINHPIPSTSIHELFAQTKRFFNLPLETKMLAKHPPQALPARGYLFVGQENVANISGYEKGLGPLKTRDIKETVDFGSANDELYDNLWVPEEELPGFRSFMEGFYELAFKTEMQLLEALAIALGVSPDHLKSLHNRAENELRILHYPAIPASELADGTATRIAEHTDFGTITMLFQDSVGGLQVEDQENLGTFNNVESASPTDIILNIGDSLQRLTNDTFKAACHRVTYPPSIKAGDGEQVIPERYSIAYFVKPNRSASLFPLKEFIEEGVPCKYEDVTAWEWNNRRIEKLFSAEAKA

SEQ ID NO: 7 - protein, 349 aa, synthetic construct
MGSHHHHHHHHGSAALNADTLDMSLFFGTPSQKQDFCDSLLRLLKKRGGVKLINHPIPSTSIHELFAQTKRFFNLPLETKMLAKHPPQALPARGYLFVGQENVANISGYEKGLGPLKTRDIKETVDFGSANDELYDNLWVPEEELPGFRSFMEGFYELAFKTEMQLLEALAIALGVSPDHLKSLHNRAENELRILHYPAIPASELADGTATRIAEHTDFGTITMLFQDSVGGLQVEDQENLGTFNNVESASPTDIILNIGDSLQRLTNDTFKAACHRVTYPPSIKAGDGEQVIPERYSIAYFVKPNRSASLFPLKEFIEEGVPCKYEDVTAEEWNNRRIEKLFSAEAKA

SEQ ID NO: 8 - protein, 349 aa, synthetic construct
MGSHHHHHHHHGSAALNADTLDMSLFFGTPSQKQDFCDSLLRLLKARGGVKLINHPIPSTSIHELFAQTKRFFNLPLETKMLAKHPPQALPARGYLFVGQENVANISGYEKGLSPLKTRDIKETVDFGSANDELYDNLWVPEEELPGFRSFMEGFYELAFKTEMQLLEALAIALGVSPDHLKSLHNRAENELRILHYPAIPASELADGTATRIAEHTDFGTITMLFQDSVGGLQVEDQENLGTFNNVESASPTDIILNIGDSLQRLTNDTFKAACHRVTYPPSIKAGDGEQVIPERYSVAYFVKPNRSASLFPLKEFIEEGVPCKYEDVTAEEWNNRRIEKLFSAEAKA

SEQ ID NO: 9 - protein, 349 aa, synthetic construct
MGSHHHHHHHHGSAALNADTLDMSLFFGTPSQKQDFCDSLLRLLKARGGVKLINHPIPSTSIHELFAQTKRFFNLPLETKMLAKHPPQALPARGYLFVGQENVANISGYEKGLSPLKTRDIKETVDFGSANDELYDNLWVPEEELPGFRSFMEGFYELAFKTEMQLLEALAIALGVSPDHLKSLHNRAHNELRILHYPAIPASELADGTATRIAEHTDFGTITMLFQDSVGGLQVEDQENLGTFNNVESASPTDIILNIGDSLQRLTNDTFKAACHRVTYPPSIKAGDGEQVIPERYSVAYFVKPNRSASLFPLKEFIEEGVPCKYEDVTAEEWNNRRIEKLFSAEAKA

SEQ ID NO: 10 - protein, 349 aa, synthetic construct
MGSHHHHHHHHGSAALNADTLDMSLFFGTPSQKQDFCDSLLRLLKARGGVKLINHPIPSTSIHELFAQTKRFFNLPLETKMLAKHPPQPLPARGYLFVGQENVANISGYEKGLSPLKTRDIKETVDFGSANDELYDNLWVPEEELPGFRSFMEGFYELAFKTEMQLLEALAIALGVSPDHLKSLHNRAHNELRILHYPAIPASELADGTATRIAEHTDFGSITMLFQDSVGGLQVEDQENLGTFNNVESASPTDIILNIGDSLQRLTNDTFKAACHRVTYPPSIKAGDGEQVIPERYSVAYFVKPNRSASLFPLKEFIEEGVPCKYEDVTAEEWNNRRIEKLFSAEAKA

SEQ ID NO: 11 - protein, 349 aa, synthetic construct
MGSHHHHHHHHGSAALNADTLDMSLFFGTPSQKQDFCDSLLRLLKARGGVKLINHPIPSTSIHELFAQTKRFFNLPLETKMLAKHPPQALPARGYSFVGQENVANISGYEKGLSPLKTRDIKETVDFGSANDELYDNLWVPEEELPGFRSFMEGFYELAFKTEMQLLEALAIALGVSPDHLKSLHNRAHNELRILHYPAIPASELADGTATRIAEHTDFGSITMLFQDSVGGLQVEDQENLGTFNNVESASPTDIILNIGDSLQRLTNDTFKAACHRVTYPPSIKAGDGEQVIPERYSVAYFVKPNRSASLFPLKEFIEEGVPCKYEDVTAEEWNNRRIEKLFSAEAKA

SEQ ID NO: 12 - protein, 349 aa, synthetic construct
MGSHHHHHHHHGSAALNADTLDMSLFFGTPSQKQDFCDSLLRLLKARGGVKLINHPIPSESIHELFAQTKRFFNLPLETKMLAKHPPQALPARGYSFVGQENVANISGYEKGLSPLKTRDIKETVDFGSANDELYDNLWVPEEELPGFRSFMEGFYELAFKTEMQLLEALAIALGVSPDHLKSLHNRAHNELRILHYPAIPASELADGTATRIAEHTDFGSITMLFQDSVGGLQVEDQENLGTFNNVESASPTDIILNIGDSLQRLTNDTFKAACHRVTYPPSIKAGDGEQVIPERYSVAYFVKPNRSASLFPLKEFIEEGVPCKYEDVTAEEWNNRRIEKLFSAEAKA

SEQ ID NO: 13 - protein, 349 aa, synthetic construct
MGSHHHHHHHHGSAALNADTLDMSLFFGTPSQKQDFCDSLLRLLKARGGVKLINHPIPSESIHELFAQTKRFFNLPLETKMLAKHPPQALPARGYAFVGQENVANISGYEKGLSPLKTRDIKETVDFGSANDELYDNLWVPEEELPGFRSFMEGFYELAFKTEMQLLEALAIALGVSPDHLKSLHNRAHNELRILHYPAIPASELADGTATRIAEHTDFGSITMLFQDSVGGLQVEDQENLGTFNNVESASPTDIILNIGDSLQRLTNDTFKAACHRVTYPPSIKAGDGEQVIPERYSVAYFVKPNRSASLFPLKEFIEEGVPCKYEDVTAEEWNNRRIEKLFSAEAKA

SEQ ID NO: 14 - protein, 349 aa, synthetic construct
MGSHHHHHHHHGSAALNADTLDMSLFFGTPSQKQDFCDSLLRLLKARGGVKLINHPIPSESIHELFAQTKRFFNLPLETKMLAKHPPQALPARGYAFVGQENVANISGYEKGLPPLKTRDIKETVDFGSANDEKYDNLWVPEEELPGFRSFMEGFYELAFKTEMQLLEALAIALGVSPDHLKSLHNRAHNELRILHYPAIPASELADGTATRIAEHTDFGSITMLFQDGVGGLQVEDQENLGTFNNVESASPTDIILNIGDSLQRLTNDTFKAACHRVTYPPSIKDGDGEQVIPERYSVAYFVKPNRSASLFPLKEFIEEGVPCKYEDVTAEEWNNRRIEKLFSAEAKA

SEQ ID NO: 15 - protein, 349 aa, synthetic construct
MGSHHHHHHHHGSAALNADTLDMSLFFGTPSQKQDFCDSLLRLLKARGVVKLINHPIPSESIHELFAQTKRFFNLPLETKMLAKHPPQALPARGYAFVGQENVANISGWEKGLPPLKTRDIKETVDFGSANDEKYDNLWVPEEELPGFRSFMEGFYELAFKTEMQLLEALAIALGVSPDHLKSLHNRAHNELRILHYPAIPASELADGTATRIAEHTDFGSITMLFQDGVGGLQVEDQENLGTFNNVESASPTDIILNIGDSLQRLTNDTFKAACHRVTWPPSIKDGDGTQVIPERYSVAYFVKPNRSASLFPLKEFIEEGVPCKYEDLTAEEWNNRRIEKLFSAEAKA

SEQ ID NO: 16 - protein, 349 aa, synthetic construct
MGSHHHHHHHHGSAALNADTLDMSLFFGTPSQKQDFCDSLLRLLKARGVVKLINHPIPSESIHELFAQTKRFFNLPLETKMLAKHPEQALPARGYAFVGQENVANISGYEKGLPPLKTRDIKETVDFGSANDEKYDNLWVPEEELPGFRSFMEGFYELAFKTEMQLLEALAIALGVSPDHLKSLHNRAHSELRILHYPAIPASELADGTATRIAEHTDFGSITMLFQDGVGGLQVEDQENLGTFNNVESASPTDIILNIGDSLQRLTNDTFKAACHRVTWPPSIKDGDGSEVIPERYSVAYFVKPNRSASLFPLKEFIEEGVPPKYEDLTAEEWNNRRIEKLFSAEAKA

SEQ ID NO: 17 - protein, 349 aa, synthetic construct
MGSHHHHHHHHGSAALNADTLDMSLFFGTPSQKQDFCDSLLRLLKARGVVKLINHPIPSESIHELFAQTKRFFNLPLETKMLAKHPEQALPARGYAFVGQENVANISGYEKGLPPLKTRDIKETVDFGSANDEKYDNLWVPEEELPGFRSFMEGFYELAFKTEMQILEALAIALGVSPDHLKSLHNRAHSELRILHYPAIPASELADGTATRIAEHTDFGSITMLFQDGVGGLQVEDQENLGTFNNVESASPTDIILNIGDSLQRLTNDTFKAACHRVTWPPSIKDGDGSEVIPERYSVAYFVKPNRSASLFPLKEFIEEGVPPKYEDLTAEEWNNRRIEKLFSAEAKA

SEQ ID NO: 18 - protein, 349 aa, synthetic construct
MGSHHHHHHHHGSAALNADTLDMSLFFGTPSQKQDFCDSLLRLLKAKGVVKLINHPIPAESIKELFAQTKRFFNLPLETKMLAKHPEQALPARGYAFVGQENVANISGYEKGLPPLKTRDIKETVDFGSANDEKYDNLWVPEEELPGFRSFMEGFYELAFKTEMQILEALAIALGVSPDHLKSLHNGAHSELRILHYPAIPASELADGTATRIAEHTDFGSITMLFQDGVGGLQVEDQENLGTFNNVESASPTDIILNIGDSLQRLTNDTFKAACHRVTWPPSIKDGDGSEVIPERYSVAYFVKPNRSASLFPLKEFIEEGVPPKYEDLTFEEWNNRRIEKLFSAEAKA

SEQ ID NO: 19 - protein, 349 aa, synthetic construct
MGSHHHHHHHHGSAALNADTLDMSLFFGTPSQKQDFCDSLLRLLKAKGVVKLINHPIPAESIKELFAQTKRFFNLPLETKMLAKHPEQALPARGYAFVGQENVANISGYEKGLPPLKTRDIKETVDFGSANDEKYDNLWVPEEELPGFRSFMEGFYELAFKTEMQILEALAIALGVSPDHLKSLHNSAHSELRILHYPAIPASELADGTATRIAEHTDFGSITMLFQDGVGGLQVEDQENLGTFNNVESASPTDIILNIGDSLQRLTNDTFRAACHRVTWPPSIKDGDGSEVIPERYSVAYFVKPNRSASLFPLKEFIEEGVPPKYEDLTFEEWNNRRIEKLFSAEAKA